(19) United States
(12) Patent Application Publication
(10) Pub. No.: US 2007/0077607 A1
(43) Pub. Date: Apr. 5, 2007

(54) Novel screening method

(76) Inventors: Dilek Telci, Birmingham (GB); Elisabetta Verderio-Edwards, Nottingham (GB); Martin Griffin, Birmingham (GB)

Correspondence Address: FISH & RICHARDSON PC, P.O. BOX 1022, MINNEAPOLIS, MN, 55440-1022, US

(21) Appl. No.: 10/544,432

(22) PCT Filed: Feb. 5, 2004
(86) PCT No.: PCT/GB04/00439

§ 371 Date: Dec. 5, 2006

(30) Foreign Application Priority Data
Feb. 5, 2003 (GB) 0304993.9

(51) Int. Cl. G01N 33/52; C12Q 1/48

(52) U.S. Cl. 435/7.92; 435/15

(57) ABSTRACT

The present invention provides a method for identifying a drug-like compound, or lead compound for the development of a drug-like compound, which is capable of modulating cellular adhesion and/or cell survival comprising the step of testing said compound for an ability to modulate the binding of a complex of tissue transglutaminase and fibronectin to a heparan sulfate containing receptor. In particular, there is provided a method for identifying a compound for use in the curative and/or prophylactic treatment of a proliferative disorder or with efficacy in promoting wound healing and /or cell survival, and compounds identified by such methods.

The present invention relates to a method for identifying compounds capable of modulating cellular adhesion and, in particular, compounds which modulate the binding of a complex of tissue transglutaminase and fibronectin to a heparan sulfate containing receptor. The present invention further relates to compounds identifiable by such methods and uses of the same.

### BACKGROUND

Adhesion of cells to the extracellular matrix (ECM) is known to provide signals controlling a number of different cell functions including that of is survival (Schlaepfer et al., 1999; Boudreau et al., 1995). When deprived of these signals most anchorage dependent cells initiate a cellular suicide programme similar to apoptosis, known as anoikis (Aplin, 1999; Frisch and Screaton, 2001).

A central component of the ECM, which regulates adhesion-dependent survival signalling, is the adhesive glycoprotein fibronectin (FN). FN binds to cell-surface matrix receptors, primarily the α5β1 integrins through the ArgGlyAsp (RGD) cell-binding domain within the III10 module. The importance of the ROD cell binding domain in adhesion-mediated cell survival has been demonstrated by employing synthetic peptides containing the RGD motif, which were found to induce proteolysis of cell survival mediators and apoptosis in many cell types, by both acting as competitive inhibitors of FN-integrin interaction and direct activators of caspase 3 (Hadden and Henke, 2000). A comparable scenario may occur in wounding and inflammatory conditions (McCawley and Matrisian, 2001), whereby fragmentation of FN elicited by the increased presence of matrix-degrading enzymes leads to apoptosis, which is induced by disruption of integrin-mediated adhesion signalling (Kapila et al. 1999). However, the RGD cell-binding domain of FN is not sufficient in isolation to regulate cell survival, which must be sustained by cell adhesion to other critical FN domains. Such an example is the C-terminal high affinity heparin-binding domain (HepII) (Jeong et al. 2001; Kapila, 2002), known to interact synergistically with heparan sulfate proteoglycan (HSPG) receptors (i.e. heparan sulfate proteoglycan receptors or heparan sulfate receptors) (Tumova et al. 2000) and with integrin α4β1 (Sharma et al. 1999). Thus, the control of anchorage-dependent cell survival by FN is a highly sophisticated process, modulated by multiple domains and involves a cross-talk between integrin and non-integrin receptors (Kapila 1999; Jeong et al. 2001; Hocking and Kowalski, 2002).

Among the different molecules that bind specialised FN domains in the extracellular space and mediate FN functions is tissue-type transglutaminase (tTG, type II). Tissue transglutaminase is a high affinity binding partner for FN and modulator of the FN matrix, which belongs to a large family of calcium dependent transamidases (Aeschlimann and Thomazy, 2000; Griffin et al. 2002; Grenard et al. 2001). It is accepted that tTG is a multifunctional protein implicated in diverse normal and pathological processes but more specifically is regarded as an important component of cell/tissue defence in response to cell damage and stress (Aeschlimann and Thomazy, 2000; Haroon et al., 1999; Johnson et al., 1999). Tissue transglutaminase differs from the other members of the family in that the transamidase active site is integrated with a GTP binding and hydrolysis site, which negatively regulates the protein transamidation activity by blocking the access to the active site via conformational changes. Another peculiarity of tTG is its externalisation into the ECM via a non-Golgi/ER route, through a mechanism which appears to depend on its active-state conformation (Balklava et al., 2002) and an intact FN binding site (Gaudry et al., 1999). The matrix deposition of tTG increases in situations of tissue damage and cellular stress (Upchurch et al., 1987, Johnson et al., 1999, Haroon et al., 1999). Once released from cells, tTG binds tightly to FN, as recently visualised by immunofluorescence and immunogold electron microscopy (Gaudry et al., 1999, Verderio et al., 1998, Verderio et al., 1999).

The involvement of tTG in cell adhesion is now consolidated (Gentile et al., 1992; Jones et al., 1997; Verderio et al., 1998; Belkin et al., 2001; Nanda et al. 2001), however the molecular mechanism underlying this process requires elucidation. It was initially thought that tTG enhances cell adhesion through matrix remodelling, via protein crosslinking (Aeschlimann and Thomazy, 2000). However, this theory has been partly obscured by recent findings that tTG is implicated in cell adhesion and motility independently from its transamidation activity (Akimov et al., 2000; Balklava et al., 2002). It has also been proposed that cell-surface tTG might act as an adhesion co-receptor of α1 and β3 integrins by mediating cell adhesion to the gelatin binding domain of FN (Akimov et al., 2000). It has also been suggested that tTG may instead act as an independent cell adhesion protein, which specifically binds to α4β1 and α9β1 integrins (Takahashi et al., 2000).

Cellular adhesion processes have been implicated in a large of number of diseases and conditions, notably proliferative disorders, inflammatory disorders and wound healing. Hence, compounds that are able to modulate cellular adhesion processes have utility in the treatment of such diseases and conditions.

### SUMMARY OF THE INVENTION

A first aspect of the present invention provides a method for identifying a drug-like compound or lead compound for the development of a drug-like compound, which compound is capable of modulating cellular adhesion and/or cell survival comprising the step of testing a compound to be tested for an ability to modulate the binding of a complex of tissue transglutaminase and fibronectin to a heparan sulfate containing receptor.

By “cell adhesion” we mean an RGD-independent cell adhesion pathway mediated by fibronectin-bound tissue transglutaminase, for example as described in the Examples below.

By “cell survival” we mean the ability of cells or tissues, in vitro or in vivo, to maintain their cellular functions.

By “a tissue transglutaminase” we mean a member of the group of enzymes identified by Enzyme Commission System of Classification No. 2.3.2.13 (EC 2.3.2.13).

Preferably, the tTG/FN complex comprises a tissue transglutaminase and/or fibronectin of mammalian, e.g. human, origin. For example, the transglutaminase and/or fibronectin may be prepared (i.e. extracted) from tissue samples or expressed by recombinant means.

It will be appreciated by persons skilled in the art that tTG and/or FN may be a wildtype, i.e. naturally occurring, tTG or FN.

Alternatively, the tTG and/or FN may be a variant of a wildtype tTG or FN, such as a fragment, which variant has an ability to form tTG/FN complexes capable of binding to a heparan sulfate containing receptor (preferably in an RGD-independent manner). By “a variant” we include a polypeptide comprising the amino acid sequence of a naturally-occurring tTG and/or FN wherein there have been amino acid insertions, deletions (i.e. fragments of a naturally-occurring tTG and/or FN) or substitutions, either conservative or non-conservative, such that the amino acid sequence changes do not substantially reduce the ability of the variant tTG and/or FN to form a tTG/FN complex capable of binding to a heparan sulfate containing receptor (preferably in an RGD-independent manner). Such variant polypeptides may be made using methods of protein engineering and site-directed mutagenesis commonly known in the art (for example, see Sambrook & Russell (2001) Molecular Cloning, A Laboratory Manual, Third Edition, Cold Spring Harbor, N.Y.

By “a heparan sulfate containing receptor” we mean transmembrane heparan sulfate proteoglycans (HSPGs) that are present on most cell types and are generally referred to as Syndecans, presently classified as Syndecans 1, 2, 3 and 4. (Woods, A. (2001). J. Clin. Invest. 107 (8), 935-941. Preferably, the heparan sulfate containing receptor is syndecan-4 (for review, see Bass & Humphries, 2002).

By “modulate” we include compounds that change, either positively or negatively, the binding of a complex of tissue transglutaminase and fibronectin to a heparan sulfate containing receptor. For example, the compound may modulate the affinity of the complex for the receptor, or vice versa. We also include compounds which modulate, either positively or negatively, the functional consequences of binding of a complex of tissue transglutaminase and fibronectin to a heparan sulfate containing receptor, for example protein kinase Cα (PKCα) activity and tyrosine phosphorylation of focal adhesion kinase (FAK) (see Examples).

In a preferred embodiment of the first aspect of the invention, the method is for identifying a drug-like compound or lead compound for the development of a drug-like compound for use in the treatment of proliferative disorders e.g. wound healing, tissue fibrosis, cancer, proliferative kidney disease IgA nephropathy. Preferably, the method is for identifying a compound with efficacy in preventing or slowing the formation of metastases.

Thus, the present invention provides a method for identifying a drug-like compound or lead compound for the development of a drug-like compound for use in the treatment of a proliferative disease comprising the step of testing said compound for an ability to modulate the binding of a complex of tissue transglutaminase and fibronectin to a heparan sulfate containing receptor.

By “efficacy in the treatment of a proliferative disorder” we include efficacy in the curative and/or prophylactic treatment of a proliferative disorder, for example an ability to prevent the onset and/or progression of said disease, and/or alleviate the symptoms of said disease.

In an alternative embodiment, the method is for identifying a drug-like compound or lead compound for the development of a drug-like compound that modulates wound healing.

Thus, the present invention further provides a method for identifying a drug-like compound or lead compound for the development of a drug-like compound for use in modulating wound healing comprising the step of testing said compound for an ability to either promote or inhibit the binding of a complex of tissue transglutaminase and fibronectin to a heparan sulfate containing receptor.

By “wound healing” we include the process of tissue repair following trauma/injury, typically comprising an inflammatory response followed by matrix deposition and eventual recellarisation of the damaged area. In skin, for example, the inflammatory response is followed by reepithelialisation of the wound area and remodelling of the granulation tissue with accompanying neovascularisation.

Compounds identified by the methods of the invention may also have utility in the treatment of other indications and conditions, such as scarring, in particular hypertrophic scarring, angiogenesis, and pancreatic β-cell function in Type II diabetes.

The term “drug-like compound” is well known to those skilled in the art, and includes a compound having characteristics that may make it suitable for use in medicine, for example as the active ingredient in a medicament. Thus, for example, a drug-like compound may be a molecule that may be synthesised by the techniques of organic chemistry, molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include a compound which, whilst not itself suitable for use as a drug (for example because it is only weakly potent against its intended target, non-selective in its action, unstable, poorly soluble, difficult to synthesise or has poor bioavailability), may provide a starting-point for the design of other compounds that may have more desirable characteristics.

In a preferred embodiment of the first aspect of the invention, the method comprises the following steps:
(a) contacting a tissue transglutaminase (tTG) with fibronectin (FR) to form a tTG/FN complex;
(b) exposing the tTG/FN complex of step (a) to a compound to be tested;
(c) exposing the treated complex of step (b) to a heparan sulfate containing receptor; and
(d) measuring the binding of the treated complex to the heparan sulfate containing receptor and/or a functional marker of such binding.

In step (a), the tissue transglutaminase and/or fibronectin may be obtained from any known source. For example, the tissue transglutaminase and/or fibronectin may be derived, i.e. extracted, from mammalian, e.g. human, tissue or cells, such as lung, liver, spleen, kidney, heart muscle, skeletal muscle, eye lens, endothelial cells, erythrocytes, smooth muscle cells, bone and macrophages. Samples.

Alternatively, the tissue transglutaminase may be obtained from a commercial source, such as guinea pig liver transglutaminase from Sigma-Aldrich (Cat. No. T5398). Likewise, fibronectin may be obtained from known sources (for example, from Sigma Aldrich).

In a further alternative embodiment, the tissue transglutaminase and/or fibronectin may be obtained by recombinant means. For example, cross-linked extracellular matrix material (ECM), comprising tTG complexed to FN, may be derived from cells capable of secreting and depositing ECM which naturally express low levels of a tissue transglutaminase (or which do not naturally express a tissue transglutaminase) but are transfected with a nucleic acid molecule encoding a transglutaminase so that expression of the enzyme is increased. Preferably, the cells are fibroblast cells, such as Swiss 3T3 fibroblast cells, transfected with a nucleic acid molecule encoding tissue transglutaminase, as described in Verderio et al. (1998) Exp. Cell Res. 239:119-38. Thus, the term ‘tTG/FN complex’ includes a cell-secreted matrix rich in tTG and FN.

Exemplary nucleotide sequences encoding tissue transglutaminases are known in the art. For example, the coding sequence for human tissue transglutaminase is disclosed in Gentile et al., 1991, J. Biol. Chem. 266(1) 478-483 (Accession no. M55153).

Nucleic acid molecules encoding a transglutaminase may be used in accordance with known techniques, appropriately modified in view of the teachings contained herein, to construct an expression vector, which is then used to transform an appropriate host cell for the expression and production of the polypeptide of the invention. Methods of expressing proteins in recombinant cells lines are widely known in the art (for example, see Sambrook & Russell, 2001, Molecular Cloning, A Laboratory Manual, Third Edition, Cold Spring Harbor, N.Y.). Exemplary techniques also include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker.

In brief, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. Thus, the DNA insert may be operatively linked to an appropriate promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome binding site for translation (e.g. see WO 98/16643).

The vector is then introduced into the host through standard techniques. Generally, not all of the hosts will be transformed by the vector and it will therefore be necessary to select for transformed host cells. One selection technique involves incorporating into the expression vector a DNA sequence marker, with any necessary control elements, that codes for a selectable trait in the transformed cell. Such markers include dihydrofolate reductase, G418 or neomycin resistance for eukaryotic cell culture. Alternatively, the gene for such a selectable trait can be on another vector, which is used to co-transform the desired host cell.

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. Examples of an inducible mammalian expression vectors include pMSG, also available from Pharmacia (Piscataway, N.J., USA), and pTet-off and pTRE2 available from Clontech (Catalogue Nos K1620-A and 6241-1, respectively, Clontech, Palo Alto, Calif., USA). The pMSG vector uses the glucocorticoid-inducible promoter of the mouse mammary tumour virus long terminal repeat to drive expression of the cloned gene. The pTet-off and pTRE2 vectors use the presence or absence of tetracycline to induce protein expression via the tet-controlled transcriptional activator.

Host cells, for example murine Swiss 3T3 cells, that have been transformed by the recombinant DNA encoding the transglutaminase, are then cultured for a sufficient time and under appropriate conditions to permit the expression of the transglutaminase and its subsequent deposition and cross-linking to form a tTG/FN-rich matrix.

It will be appreciated by persons skilled in the art that tTG and/or FN may be a wildtype, i.e. naturally occurring, tTG or FN. Preferably, the tTG and/or FN are mammalian, e.g. human, in origin.

Alternatively, the tTG and/or FN may be a variant of a wildtype tTG or FN, such as a fragment, which variant has an ability to form tTG/FN complexes capable of binding to a heparan sulfate containing receptor (preferably in an RGD-independent manner).

Step (b) comprises contacting the tTG/FN complex of step (a) with a compound to be tested. Alternatively, the compound to be tested may be incubated with tTG and/or FN prior to the formation of the complex in step (a).

Preferably, the compound to be tested is used at a range of doses and/or for various incubation times.

Step (c) comprises contacting the treated complex of step (b) with a heparan sulfate containing receptor or a fragment thereof which has an ability to bind to tTG/FN complexes (preferably in an RGD-independent is manner). In a preferred embodiment, the heparan sulfate containing receptor is syndecan-4 (see Bass & Humphries, 2002), or a fragment thereof which has an ability to bind to tTG/FN complexes (preferably in an RGD-independent manner).

Preferably, the heparan sulfate containing receptor is expressed on the surface of a cell. For example, cells may be used which naturally express a heparan sulfate containing receptor. Alternatively, cells may be used which do not naturally express a heparan sulfate containing receptor but which have been engineered by recombinant means to express such a receptor. Such recombinant methods are well known in the art (for example, see Sambrook & Russell, supra).

Suitable cells include primary human osteoblasts (HOB), which can be isolated from explants of trabecular bone dissected from femoral heads (see Examples), Swiss 3T3 fibroblasts and the human bladder carcinoma cell line ECV304 (obtainable from ATCC).

Step (d) comprises measuring the binding of the treated complex to the heparan sulfate containing receptor and/or a functional marker of such binding. Preferably, binding is compared to that observed in a control untreated sample, for example between the tTG/FN complex and the heparan sulfate containing receptor in the absence of treatment with the compound to be tested, a difference in binding being indicative of modulation of cellular adhesion and/or cell survival.

Suitable methods of measuring or detecting the binding of the tTG/FN complex to the heparan sulfate containing receptor will be apparent to those skilled in the art.

In a preferred embodiment of the first aspect of the invention, step (d) comprises using an enzyme-linked immunosorbent assay (ELISA). Such methodology is well known in the art (for example, see Sblattero et al, 2000, Am. J. Gastroenterol. 95:1253-57). In ELISA assays, the binding of a (primary) antibody to a target antigen is detected by means of a secondary antibody with affinity for the primary antibody. The secondary antibody is conjugated to an enzyme, such as horseradish peroxidase, which catalyses the transformation of a non-detectable substrate to a detectable product. Thus, the detectable product gives a measure of the binding of the primary antibody. Often, the detectable product is coloured and may be detected by spectrophotometry.

Alternatively, a surface plasmon resonance assay, for example similar to that described in Plant et al (1995) Analyt Biochem 226(2), 342-348, may be used.

Methods may also make use of a polypeptide (or compound) that is labelled, for example with a radioactive or fluorescent label.

In a preferred embodiment, RGD-dependent binding is inhibited in step (d), for example by incubating the tTG/FN complex and heparan sulfate containing receptor in the presence of RGD-containing peptide, such as GRGDTP.

The method used in step (d) may be capable of high throughput operation, for example a chip-based method, for example in which the compounds to be tested are immobilised in a microarray on a solid support, as known to those skilled in the art.

Further examples may include cell based assays and protein-protein binding assays. An SPA-based (Scintillation Proximity Assay; Amersham International) system may be used. Conveniently this is done in a 96-well format. Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

The yeast two-hybrid system may also be used, as well known to those skilled in the art, Fields & Song, Nature 340:245-246 (1989).

Optionally, the binding of the tTG/FN complex to the heparan sulfate containing receptor is determined indirectly by measuring a functional marker of such binding, such as:
(a) Cell attachment;
(b) Cell spreading;
(c) Formation of focal adhesion structures, e.g. by cytoskeletal staining;
(d) PKCα activity; and
(e) FAK activity.

Such indirect methods are described in detail in the Examples.

In an alternative preferred embodiment of the methods of the first aspect of the invention, the method comprises the following steps:
(a) contacting tTG with FN to form a tTG/FN complex;
(b) exposing a heparan sulfate containing receptor to a compound to be tested;
(c) exposing the tTG/FN complex of step (a) to the treated heparan sulfate containing receptor of step (b); and
(d) measuring the binding of the treated complex to the heparan sulfate containing receptor and/or a functional marker of such binding.

In a further alternative preferred embodiment of the methods of the first aspect of the invention, the method comprises the following steps:
(a) contacting tTG with FN to form a tTG/FN complex;
(b) exposing the tTG/FN complex of step (a) to a heparan sulfate containing receptor;
(c) exposing the tTG/FN complex and heparan sulfate containing receptor of step (b) to a compound to be tested; and
(d) measuring the binding of the treated complex to the heparan sulfate containing receptor and/or a functional marker of such binding.

Test compounds that modulate the binding of the tTG/FN complex to the heparan sulfate containing receptor in step (d) are identified as candidate compounds for modulating cell adhesion and/or cell viability, e.g. for use in the treatment of proliferative disorders, wound healing, etc.

In a preferred embodiment of the methods of the first aspect of the invention, the tTG/FN complexes or heparan sulfate containing receptors are adapted to render the complexes immobilised to a 96 well plate or other suitable vessel for drug screening. Thus, the above methods can be adapted such that the tTG/FN complex, or receptor, is linked (directly or indirectly) to the plate. Molecules which are not associated with such complexes are then removed, e.g. by washing. Changes in binding of the complex to the receptor can be tested for by ELISA techniques, i.e. by binding of antibody to target and observation of bound antibody using enzyme linked secondary antibody (see below).

It will be appreciated that screening assays which are capable of high throughput operation will be particularly preferred. For example, an SPA-based (Scintillation Proximity Assay; Amersham International) system may be used.

Further methods capable of high throughput operation include chip-based methods. For example, new technology, called VLSIPS™, has enabled the production of extremely small chips that contain hundreds of thousands or more of different molecular probes. These biological chips or arrays have probes arranged in arrays, each probe assigned a specific location. Biological chips have been produced in which each location has a scale of, for example, 10 μm. The chips can be used to determine whether target molecules interact with any of the probes on the chip. After exposing the array to target molecules under selected test conditions, scanning devices can examine each location in the array and determine whether a target molecule has interacted with the probe at that location.

The purpose of the screening method of the first aspect of the invention is to identify (and select for further investigation) compounds which may be useful as modulators of cell adhesion and/or cell viability. The condition (i.e. the required binding of the tTG/FN complex to the heparan sulfate containing receptor or required change in the ability of the tTG/FN complex to bind to the heparan sulfate containing receptor) which the compound must satisfy in order to be identified as a drug-like compound or lead compound for the development of a drug-like compound may be set at a value (expressed, for example, as a binding or dissociation constant) achieved by compounds capable of achieving the required change in the binding of the tTG/FN complex to bind to the heparan sulfate containing receptor.

Preferably, the compound is identified as a positive modulator if it increases binding of a complex of tissue transglutaminase and fibronectin to a heparan sulfate containing receptor by at least 10% compared to binding in a control sample which has not been treated with the compound to be tested. More preferably, the compound is identified as a positive modulator if binding is increased at least 20%, 30%, 40%, 50%, 60%, 70%, 80%, 90%, 100%, 150%, 200%, 300%, 400%, 500% or 1000% compared to controls.

Such positive modulators of binding of a tTG-FN complex to a heparan sulfate containing receptor may be used for the following:
(i) to promote wound healing and prevent extensive cell death following tissue damage/trauma, e.g. following burns;
(ii) to promote wound healing where, through disease or other abnormality, it is hindered or does not take place e.g. in diabetic ulcers, bed sores etc;
(iii) to stabilise primary cell cultures and or organ cultures, e.g. pancreatic β-cells following isolation; and
(iv) to prevent or inhibit tumour growth (in some instances Syndecans are reduced in cancer).

Likewise, the compound is preferably identified as a negative modulator if it decreases binding of a complex of tissue transglutaminase and fibronectin to a heparan sulfate containing receptor by at least 10% compared to binding in a control sample which has not been treated with the compound to be tested. More preferably, the compound is identified as a positive modulator if binding is decreased at least 20%, 30%, 40%, 50%, 60%, 70%, 80%, 90%, 95%, 96%, 97%, 98%, 99% or 100% compared to controls. Most preferably, the compound substantially inhibits binding of a complex of tissue transglutaminase and fibronectin to a heparan sulfate containing receptor.

Such negative modulators of binding of a tTG-FN complex to a heparan sulfate containing receptor may be used for the following:
(i) to prevent or inhibit tumour growth;
(ii) to prevent or inhibit metastases;
(iii) to prevent or inhibit tissue scarring; and
(iv) to prevent or inhibit tissue fibrosis.

In a further preferred embodiment of the method of the invention, a pre-screening step is included wherein, prior to testing a compound to be tested for an ability to modulate the binding of a complex of tissue transglutaminase and fibronectin to a heparan sulfate containing receptor (e.g. prior to step ‘a’), a prospective test compound is tested to determine if it binds to tissue transglutaminase and/or fibronectin, either individually or in a complex (in the absence of a heparan sulfate containing receptor) and/or if it binds to a heparan sulfate containing receptor (in the absence of a complex of tissue transglutaminase and fibronectin). Compounds which exhibit binding to one or more of these individual components in such a pre-screen may then be selected as test compounds for the full screening method.

Advantageously, prospective compounds are selected which bind more strongly to a complex of tissue transglutaminase and fibronectin than to either individual component of the complex.

It will be understood that it is desirable to identify compounds that may modulate binding of a complex of tissue transglutaminase and fibronectin to a heparan sulfate containing receptor in vivo. Thus, it will be understood that reagents and conditions used in the methods of the invention may be chosen such that the interactions between said complex of tissue transglutaminase and fibronectin and the heparan sulfate containing receptor are substantially the same as would occur iii vivo.

Advantageously, the results of the methods are furnished in an intelligible format. Preferably, the results are recorded or stored on an information carrier. However, the step of finishing the results could be by communicating the results orally.

By “information carrier”, we include any means of storing information, such as paper, a computer disk; an internet-based information transfer system, such as an e-mail or internet page, or electronic file, etc. Of course, an “intelligible format” is also intended to embrace encrypted information which can be deciphered with an approximate key.

It will be appreciated by persons skilled in the art that lead compounds identified by the screening method of the invention may be developed further, for example by molecular modeling/and or experiments to determine the structure activity relationship in order to develop more efficacious compounds, for example by improving potency, selectivity/specificity and pharmacokinetic properties.

A second aspect of the invention provides a kit of parts for use in the screening method of the first aspect of the invention, the kit comprising
(a) a substrate, e.g. surface, upon which tTG/FN complexes are immobilised or means for producing such as surface;
(b) a heparan sulfate containing receptor (for example, cells capable of expressing such a receptor); and
(c) means for detecting the binding of tTG/FN complexes to the heparan sulfate containing receptor.

Preferably, the kit comprises a surface upon which tTG/FN complexes are immobilised.

Conveniently, the tTG/FN complexes are derived from cells which secrete and deposit ECM and which express a tissue transglutaminase. Thus, the kits of the invention may comprise a surface to which tTG/FN complexes may be adhered and cells capable of expressing such complexes, either endogenously or as a result of transfection (such as those identified above). Prior to using the kits, the cells are cultured in contact with the surface so as to deposit cross-linked ECM material on said surface.

Alternatively, cells which naturally express low levels of a tissue transglutaminase (or which do not naturally express a tissue transglutaminase) may be cultured and an exogenous tissue transglutaminase added in order to effect cross-linking of the ECM. Thus, in one embodiment, the kits of the invention may comprise a surface to which cross-linked ECM material may be adhered, cells which naturally express low levels of a tissue transglutaminase (or which do not naturally express a tissue transglutaminase), and an exogenous tissue transglutaminase. The exogenous tissue transglutaminase may be derived from natural sources (e.g. human tissue or cells such as lung, liver, spleen, kidney, heart muscle, skeletal muscle, eye lens, endothelial cells, erythrocytes, smooth muscle cells, bone and macrophages) or may be produced by recombinant means (as described above).

It will be appreciated by persons skilled in the art that, in the methods and kits of the invention, the surface to which the cross-linked ECM material is adhered (or is to be adhered) may take one of a number or forms. For example, the surface may be interior surface of a test tube or vial or the like. Alternatively, the surface may be concave surface of a well in a single- or multi-well plate, for example a microtitre plate.

Preferably, the surface is a multi-well plate such as a 96 well, flat bottomed, tissue culture treated plate (Product code M9780, Sigma Aldrich Company Ltd, Fancy Road, Poole, Dorset BH12 4QH ,UK).

According to a third aspect of the present invention, there is provided a compound identifiable or identified by a method of the first aspect of the invention.

It will be appreciated that any compound according to the third aspect of the invention should be sufficiently non-toxic to allow use of the compound at a therapeutic dose.

A fourth aspect of the invention provides a pharmaceutical formulation comprising a compound according to the third aspect of the invention in admixture with a pharmaceutically or veterinarily acceptable adjuvant, diluent or carrier.

Preferably, the formulation is a unit dosage containing a daily dose or unit, daily sub-dose or an appropriate fraction thereof, of the active ingredient

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrasternally, intracranially, intramuscularly or subcutaneously, or they may be administered by infusion techniques. They are best used in the form of a sterile aqueous solution, which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 to 1000 mg per adult (ie. from about 0.015 to 15 mg/kg), administered in single or divided doses.

Thus, for example, the tablets or capsules of the compound of the invention may contain from 1 mg to 1000 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains at least 1 mg of a compound of the invention for delivery to the patient. It will be appreciated that he overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

Conveniently, the formulation is a pharmaceutical formulation. Advantageously, the formulation is a veterinary formulation.

A fifth aspect of the invention provides a compound according to the third aspect of the invention for use in medicine.

Preferably, the compound is for use in the curative or prophylactic treatment of proliferative disorders (e.g. inhibiting or preventing tumour growth and metastases), modulating wound healing, prevention of tissue scarring and fibrosis, stabilising primary cell cultures or organ cultures, modulation of neurodegenerative diseases and/or in slowing or preventing onset of Type II diabetes.

A sixth aspect of the invention provides the use of a compound according to the third aspect of the invention in the preparation of a medicament for in the curative or prophylactic treatment of proliferative disorders.

A seventh aspect of the invention provides the use of a compound according to the third aspect of the invention in the preparation of a medicament for modulating wound healing.

An eighth aspect of the invention provides a method for treating a patient, either prophylactically or curatively, having a proliferative disorder, the method comprising administering to the patient a compound according to the third aspect of the invention or a formulation according to the second aspect of the invention.

By ‘curatively’ treating, we mean treating a patient having an inflammatory or proliferative condition or disorder such that the symptoms and/or underlying cause(s) of the condition or disorder are alleviated, in whole or in part

In a preferred embodiment of the methods of the invention, the patient has a lympho-proliferative disorder (lymphoma), a haematological malignancy (such as leukaemia), a myeloid malignancy (myeloma), cancer of the breast, prostate or bowel or other epithelial carcinomas, cancer of the bone or neuronal tissue.

A ninth aspect of the invention provides a method for modulating wound healing in a patient, the method comprising administering to the patient a compound according to the third aspect of the invention or a formulation according to the second aspect of the invention.

In a preferred embodiment of the uses and methods of the invention, a compound of the third aspect of the invention is administering to the patient together with one or more additional medicaments. For example, the compounds of the invention can be used in the treatment of proliferative disorders in combination with existing anti-cancer agents.

It will be appreciated by persons skilled in the art that the compounds of the present invention have utility in both the medical and veterinary fields. Thus, the methods of the invention may be used in the treatment of both human and non-human animals (such as horses, dogs and cats).

A tenth aspect of the invention provides a method for making a medicament for use in modulating cell adhesion and/or cell survival (for example, for use in the treatment of a proliferative disorder, wound healing, etc) comprising identifying a compound which is able to modulate binding of a complex of tissue transglutaminase and fibronectin to a heparan sulfate containing receptor using a method according to the first aspect of the invention and providing said compound in a pharmaceutical formulation.

An eleventh aspect of the invention provides use of a tissue transglutaminase/fibronectin complex for promoting cell survival, either in vivo or in vitro.

By “a tissue transglutaminase/fibronectin complex” we mean a complex comprising a tissue transglutaminase and fibronectin wherein the components of the complex are maintained in contact with each other by non-covalent and/or covalent interactions

The tTG and FN components of the complex may be obtained from any of any known source, as described above, for example from tissue samples or by recombinant expression.

It will be appreciated by persons skilled in the art that tTG and/or FN may be a wildtype, i.e. naturally occurring, tTG or FN. Preferably, the tTG and/or FN are mammalian, e.g. human, in origin.

Alternatively, the tTG and/or FN may be a variant of a wildtype tTG or FN, such as a fragment, which variant has an ability to form tTG/FN complexes capable of binding to a heparan sulfate containing receptor (preferably in an RGD-independent manner). By “a variant” we include a polypeptide comprising the amino acid sequence of a naturally-occurring tTG and/or FN wherein there have been amino acid insertions, deletions (i.e. fragments of a naturally-occurring tTG and/or FN) or substitutions, either conservative or non-conservative, such that the amino acid sequence changes do not substantially reduce the ability of the variant tTG and/or FN to form a tTG/FN complex capable of binding to a heparan sulfate containing receptor (preferably in an RGD-independent manner). Such variant polypeptides may be made using methods of protein engineering and site-directed mutagenesis commonly known in s the art (for example, see Sambrook & Russell 2001, Molecular Cloning, A Laboratory Manual, Third Edition, Cold Spring Harbor, N.Y.).

Complexes of tissue transglutaminase and fibronectin may be formed by contacting the tTG with FN using the following exemplary protocol:

Surfaces to be coated with the complex are first coated with FN solution (for example, a 1-20 μg/m FN solution, preferably 5 μ/ml) in suitable buffer (for example, 50 mM Tris-HCl 7.4) by incubation at 4° C. for 8-15 h. The excess FN solution is removed from the coated cell surface, washed once in suitable buffer (for example, 50 mM Tris-HCl pH 7.4) and then coated with tTG solution, such as purified tTG from human or other mammalian source (for example, guinea pig liver tTG) in phosphate buffered saline (PBS) pH 7.4 containing 2 mM EDTA. An optimal concentration of the tTG for coating could be (20 μg/ml). After a one-hour incubation at 37° C., the tTG solution is removed and coated surfaces washed once in 50 mM tris-HCl pH 7.4. Prior to cell seeding the coated surface is washed once in serum free cell culture medium, e.g. Dulbecco's modified Eagles medium (DMEM), depending on the cells to be seeded.

Complexes of tTG and FN may also include a cell secreted tTG/FN rich matrix. Cells capable of secreting such a matrix include Swiss 3T3 cells transfected with tTG under the control of the tetracycline regulatable promoter and capable of inducible expression of tTG (Verderio et al, 1998). Other suitable cells include human endothelial cells and human bladder carcinoma ECV304 cells, which also express high levels of tTG.

To obtain the tTG/FN rich matrix, cells are first cultured for up to 3 weeks in suitable medium (plus 10% fetal calf serum) in order to lay down the tTG/FN rich extracellular matrix. Cells are then removed by using preferably a non-cell disruptive agent, e.g. 5 mM EDTA, in PBS pH 7.4. The remaining tTG/FN rich matrices are then washed in serum free cell culture medium (e.g. DMEM) depending on the cells to be seeded on to the matrix.

Alternatively, the tTG/FN complex can be made with both proteins in solution, e.g. 5 μg/ml FN and 20 μg/ml tTG in 20 mM Tris-HCl buffer pH 7.4 at 37° C. for 1 to 4 hours in the presence of 2 mM EDTA. The is complex is then coated onto the surface and left at 4° C. for 8-15 h and then washed once with the same Tris-HCl buffer. Prior to seeding the cells the coated surfaces are washed once in suitable (depending on cell type) serum free medium.

The complexes are then exposed to the cells in suitable cell culture medium, e.g. DMEM, depending on the cells to be used, preferably at 37° C. in a 5% CO₂:95% air atmosphere for periods of time between 1 to 72 h.

For in vivo applications, the tTG/FN complexes may be delivered to the target cells by known methods, e.g. by local injection.

In a preferred embodiment of the eleventh aspect of the invention, the tTG/FN complex is used for promoting survival of a primary cell culture.

In an alternative embodiment, the tTG/FN complex is used for maintaining the stability and maintenance of function of small organ culture.

Preferably, the tTG/FN complex is used for promoting survival of pancreatic β-cells or pancreatic islets, e.g. for use in transplants.

Thus, the invention further provides a method for promoting cell survival by exposing the cells to a transglutaminase/fibronectin complex.

Preferred, non-limiting examples which embody certain aspects of the invention will now be described, with reference to the following figures:

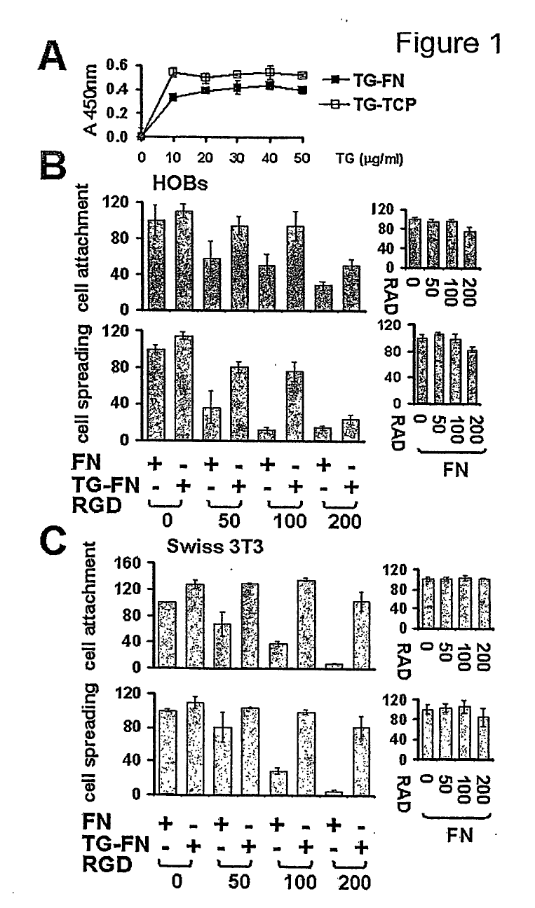

FIG. 1: Tissue transglutaminase bound to FN supports RGD-independent cell adhesion.

(A) Relative levels of tTG bound to coated FN at increasing concentrations of free tTG. Detection of tTG was performed by an ELISA-type assay as described in the Methods. Each value represents the mean±SD of three replicas from a representative experiment and data are expressed as absorbance values at 450 nm. Background absorbance (in the range of 0.02-0.15) was subtracted from data. The level of tTG bound to FN (TG-FN) after incubation with 20-μg/ml free tTG is significantly different (p<0.05) from the level after incubation with 10 μg/ml, but not 30, 40 and 50 μg/ml tTG. The level of tTG bound to tissue culture plastic (TG-TCP) represents the positive experimental control. RGD-independent cell adhesion of HOB cells (B) and Swiss 3T3 fibroblasts (C) in response to tTG-FN. Cell attachment (upper graphs in B and C) and cell spreading (lower graphs in B and C) on FN and tTG-FN was assessed after pre-incubation of cells with increasing concentrations of RGD synthetic peptides (0-200 μg/ml) (RGD 0, 50, 100, 200), as described in the Methods. Insets in B and C shows cell attachment (upper insets) and spreading (lower insets) on FN when cells were pre-incubated with control RAD peptide, at concentrations equivalent to the RGD peptide (RAD 0, 50, 100, 200). Each point represents the mean number of attached cells (cell attachment) or the mean percentage of spread cells (cell spreading)±SD, of triplicate wells from one of at least three representative experiments. Data are expressed as percentage of control values on FN, which represents 100%. HOB cells in B: mean attachment values±SD on FN control were 306±53 in upper graph, 246±13 in upper inset; mean percentage values of spread cells on FN control were 78±5 in lower graph and 82±2 in lower inset; total cells analysed in control sample were ˜900. Swiss 3T3 cells in C: mean attachment values±SD on FN control were 166±3 in upper graph, 155±14 in upper inset; mean percentage values of spread cells on FN control were 88±1 in lower graph and 86±3 in lower inset; total cells analysed in control sample were ˜500.

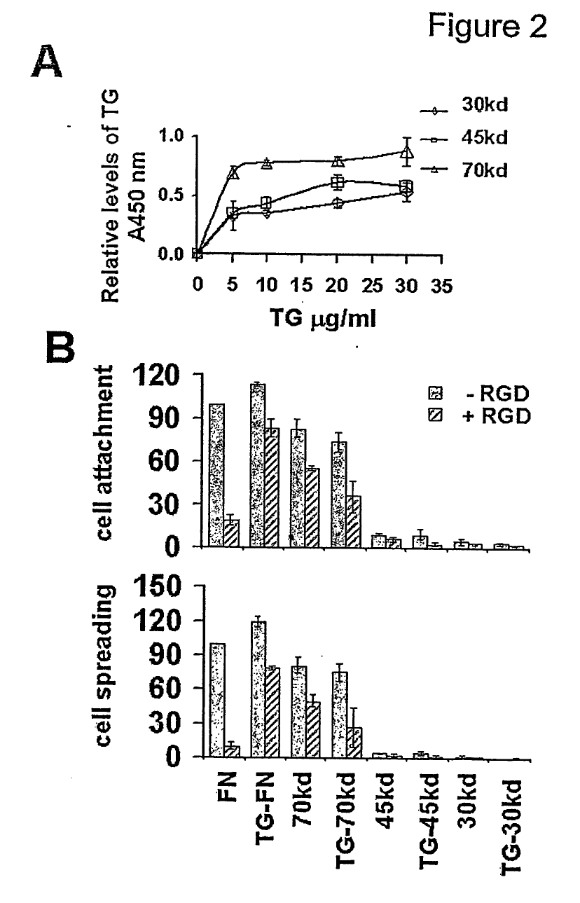

FIG. 2: tTG immobilisation on amino-terminal FN fragments is not sufficient to mediate RGD-independent cell adhesion.

(A) Relative levels of tTG bound to the amino-terminal FN fragments (70 kd, 45 kd or 30 kd) immobilised on TCP, at increasing concentrations of free tTG. Each value represents the mean±SD of three replica and data are expressed as absorbance values at 450 nm. The level of tTG bound to each fragments after incubation with 20 μg/ml tTG solution was not significantly different (p>0.05) from the level of tTG bound using 30 μg/ml tTG solution. (B) Assessment of cell adhesion in response to tTG bound to amino-terminal FN fragments. Tissue culture wells were pre-coated with amino-terminal FN fragments and in half of the wells 20 μ/ml tTG was further immobilised upon them (TG-70, TG-45, TG-30). Cell attachment (B upper) and cell spreading (B lower) of HOB cells pre-incubated with RGD peptide (100 μg/ml) or DMEM was assessed and expressed as described in legend of FIG. 1. In B upper, ordinate represents mean cell attachment expressed as mean percentage of control attachment to FN (which represents 100%)±SD of three independent experiments undertaken in triplicate (mean attachment value±SD on FN in the three experiments was 406±72, 370±49 and 465±51; total cells analysed in control samples were respectively ˜1200, ˜1100, ˜1400). In B lower, ordinate represents the mean percentage of spread cells, expressed as mean percentage of control attachment to FN (which represents 100%)±SD of three independent experiments undertaken in triplicate (mean percentage of spreading±SD on FN in the three experiment was 87±1, 91±1, 88±7.)

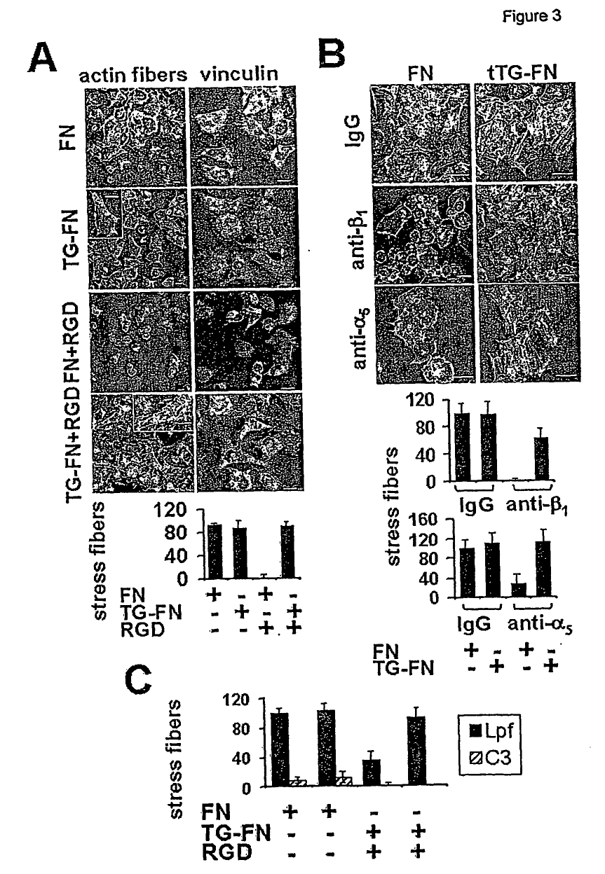

FIG. 3: Confocal laser fluorescence microscopy of RGD-independent actin cytoskeleton reorganisation and focal adhesion in response to tTG-FN.

(A) Visualisation of actin stress fibres and focal adhesions in the presence of RGD peptide. Actin stress fibres formed on FN or tTG-FN in the presence of absence of RGD peptide were detected on fixed and permeabilised cells using FITC-labelled phalloidin (actin fibres). Focal adhesions were revealed by indirect immunofluorescent staining for vinculin with a secondary antibody conjugated to FITC (vinculin). Images were acquired by confocal laser fluorescence microscopy, as described in the Methods. Cells in random fields (at least 100 cells in the control FN) were scored for actin stress fibre formation on confocal images, as outlined in the Methods, and data shown in the histogram. Ordinate represents the percentage of cells with formed stress fibres, expressed as percentage of control stress fibres in response to FN, which represents 100% (mean percentage value±SD was 74±2). The arrows point at cells magnified in the insets. Bars, 10 μm. (B) Visualisation of actin stress fibres in the presence of function blocking anti-integrin antibodies towards α5 and β1 subunits. HOB cells in suspension were pre-incubated with function blocking anti-integrin antibodies P1D6 (anti-α5) and JB1A (anti-β1) or control mouse IgGs (IgG). Cells were then seeded in the presence of the antibodies on either FN or tTG-FN and then actin stress fibres detected as described in A. The histogram shows the relative measurement of formed actin stress fibres. Cells in random fields (at least 200 cells in the control FN/IgG) were scored for actin stress fibre formation using confocal images and data were expressed as described in A (mean percentage value of actin fibres formed on control FN±SD was 49±8.4 in B upper and 53±7.8 in B lower graph). Bars, 10 μm. (C) Relative measurement of actin stress fibres following pre-incubation with RhoA inhibitor C3 exotransferase. Monolayers of HOB cells were loaded with C3 exotransferase (C3), using lipofectin reagent as delivery agent, or lipofectin only (Lpf), as described in the Methods. Before plating, (indicated cells) were pre-treated with RGD peptide (100 μg/ml). Actin stress fibres which formed on FN or tTG-FN with and without C3-exotransferase treatment, were detected and quantified as described in A. Cells in ten random fields (at least 250 cells in the control FN/Lpf) were scored for actin stress fibre formation Mean percentage value of actin fibres formed on control FN±SD was 63±5.

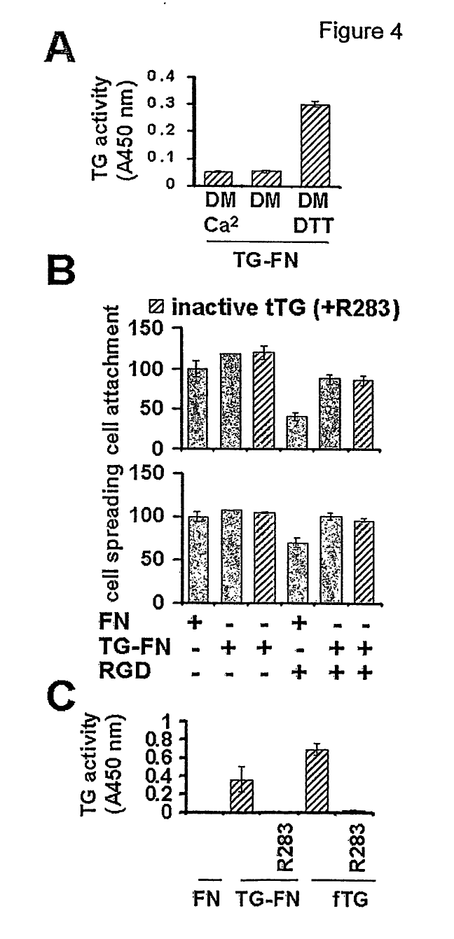

FIG. 4: tTG cross-linking activity is not required to support RGD-independent cell adhesion.

(A) Cross-linking activity of FN-bound tTG. The activity of tTG once immobilised on FN (TG-FN) was measured by assaying the level of incorporation of biotinylated cadaverine into FN in either culture medium DMEM (DM), or DMEM with 5 mM Ca²⁺ (DM-Ca²⁺) or DMEM supplemented with 5 mM DTT (DM-DTT). Data are expressed as absorbances at 450 nm (tTG activity). Each value represents the mean absorbance±SD of three replicas from a representative experiment. (B) Assessment of cell adhesion to tTG-FN following inactivation of tTG. The tTG-FN matrix was incubated with the tTG active-site inhibitor R283 (100 μM), and then utilised as adhesive substrate for HOB cells, pre-treated or not with RGD peptide (100 μg/ml), and further supplemented with the same concentration of the tTG inhibitor R283. For comparison, replica cell samples of identically treated cells were seeded on tTG-FN without R283 treatment. Cells were examined for cell attachment (B upper) and cell spreading (B lower) as described before. Each point represents the mean number of attached cells (cell attachment) or the mean percentage of spread cells (cell spreading)±SD, of triplicate wells of a representative experiment. Data are expressed as percentage of control values on FN, which represents 100% (mean attachment values ±SD were 373±36 in B upper; mean percentage values of spread cells were 89±6 in B lower; total cells analysed in control sample were ˜1100). (C) Inhibition of the cross-linking activity of FN-bound tTG (TG-FN) and tTG free in solution (fTG) by 100 μM R283, when measured via incorporation of biotinylated cadaverine into FN in buffer containing DTT. Each value represents the mean±SD of three replicas from a representative experiment undertaken in triplicate.

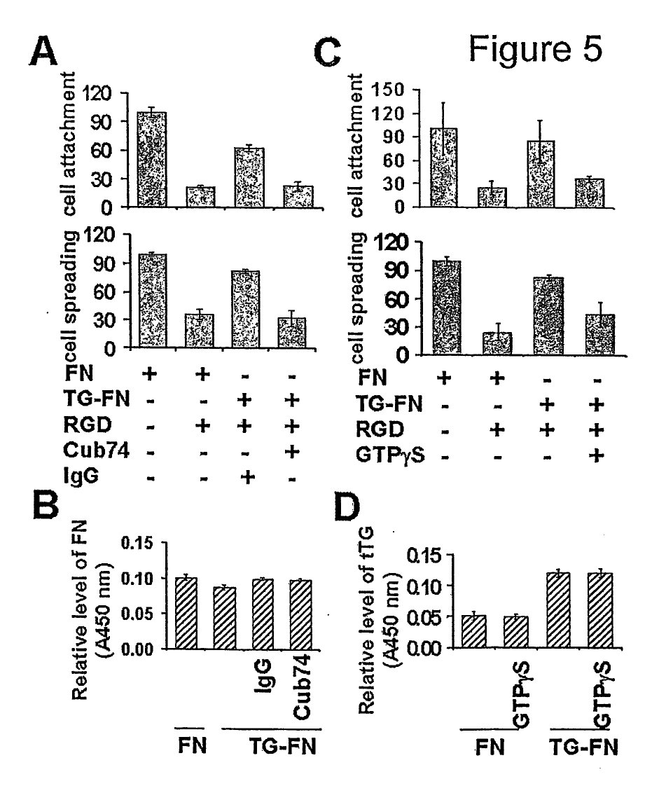

FIG. 5: RGD-independent cell adhesion in response to FN-bound tTG depends on tTG accessibility and GTP-mediated conformational change.

(A) FN-bound tTG was blocked by incubation with Cub74 (40 μg/ml) for one hour at 37° C. in PBS with 2 mM EDTA using mouse IgG1k as control antibody. After removing the unbound antibody HOB cells, pre-incubated with RGD peptides (100 μg/ml), were seeded on the tTG-FN matrix. Adhered cells were processed and examined for cell attachment (A upper) and cell spreading (A lower) (mean attachment values±SD were 229±13 in C upper; mean percentage values of spread cells were 93±2 in C lower; total cells analysed in control sample were ˜700). (B) Availability of FN, detected by an ELISA-type assay described in the Methods, after blocking the tTG-FN matrix by Cub 74 and IgG1k, in conditions identical to those used in the cell adhesion experiment shown in A. Each value represents the mean±SD of four replica from a representative experiment and data are expressed as absorbance at 450 nm. (C) HOB cells in suspension, pre-incubated with RGD peptides (100 μg/ml), were seeded in the presence of 1 mM GTP-γS on fibronectin-bound tTG, previously incubated with GTP-γS in PBS pH 7.4, for 10 minutes at room temperature. Adhered cells were processed and examined for cell attachment (C upper) and cell spreading (C lower) with and without inactivation of tTG by GTP-γS (mean attachment values±SD were 294±98.32 in C upper; mean percentage values of spread cells were 84.9±4 in C lower; total cells analysed in control sample were ˜800). (D) Relative levels of tTG that bind FN in the presence of 1 mM GTP-γS, in conditions identical to the cell adhesion experiment described in C. tTG was detected by an ELISA-type assay described in the Methods. Each value represents the mean±SD of four replicas from a representative experiment, with data expressed as absorbance at 450 nm.

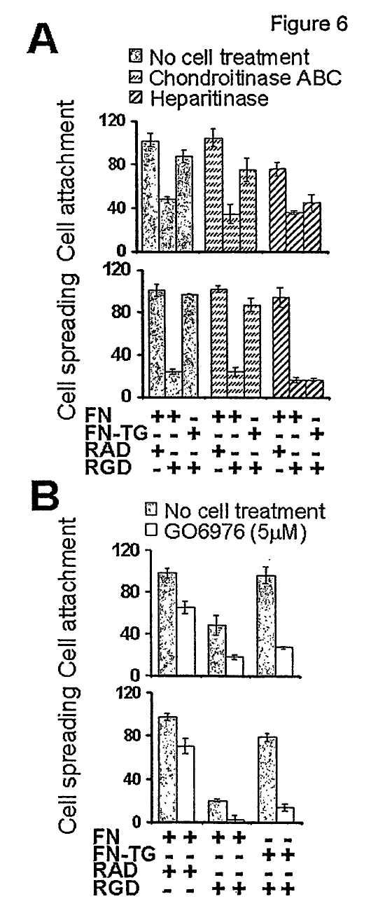

FIG. 6: RGD-independent cell adhesion to tTG-FN is dependent on cell surface heparan sulfate and PKCα activity.

(A) HOB cells in suspension (2×10⁵ cell/ml) were pre-treated with 15 mU/ml heparitinase or 15 mU/ml protease-free chondroitinase ABC in serum-free medium for one hour at 37° C. before evaluating cell attachment (upper) and cell spreading (lower) on FN and tTG-FN in the presence of RGD peptide or RAD control peptide (100 μg/ml). Each point represents the mean number of attached cells (cell attachment) or the mean percentage of spread cells (cell spreading)±SD, of triplicate wells from one of three representative experiments. Data are expressed as percentage of control values on FN, which represents 100%. Mean attachment values±SD on FN control without peptide (not shown) were 140±16; mean percentage values of spread cells on FN control were 89±2; total cells analysed in control sample were ˜420. (B) Monolayers of sub-confluent HOB cells were incubated in serum-free DMEM medium supplemented with the PKCα inhibitor GO6976 (5 μM, dissolved in DMSO) for 30 minutes or with an equal volume of DMSO only. After that cells were harvested for analysis of cell attachment (upper) and spreading (lower) on FN and tTG-FN in the presence of RGD peptide (100 μg/ml) or RAD control peptide (100 μg/ml) as described above. Where indicated, 5 μM GO6976 was included in the culture medium in all steps throughout the adhesion assay. Data are expressed as percentage of control values on FN, which represents 100%. Mean attachment values±SD on FN control without peptide (not shown) were 197±8; mean percentage values of spread cells on FN control were 83±4; total cells analysed in control sample were ˜590.

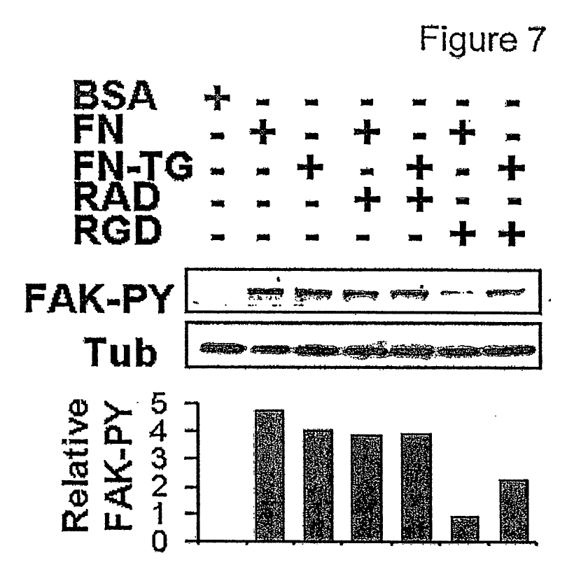

FIG. 7: RGD-independent adhesion in response to fibronectin-bound tTG promotes tyrosine phosphorylation of FAK

Lysates of HOB cells grown in the presence of RGD peptide (100 μg/ml) or control RAD peptide (100 μ/ml), on FN or tTG-FN, were western blotted and then phosphorylated tyrosine FAK (FAK-PY) revealed by probing with phospho(Tyr397)-FAK antibody, as described in the Methods. After stripping, blots were re-probed to reveal control protein tubulin (Tub). Density of bands was quantified by scanning densitometry. Net area values for phospho-FAK bands were normalised against values for tubulin bands, taking the tubulin net area value of the BSA sample as normalising value, and expressed as relative FAK phosphorylation (histogram). Results from one of three typical experiments are shown.

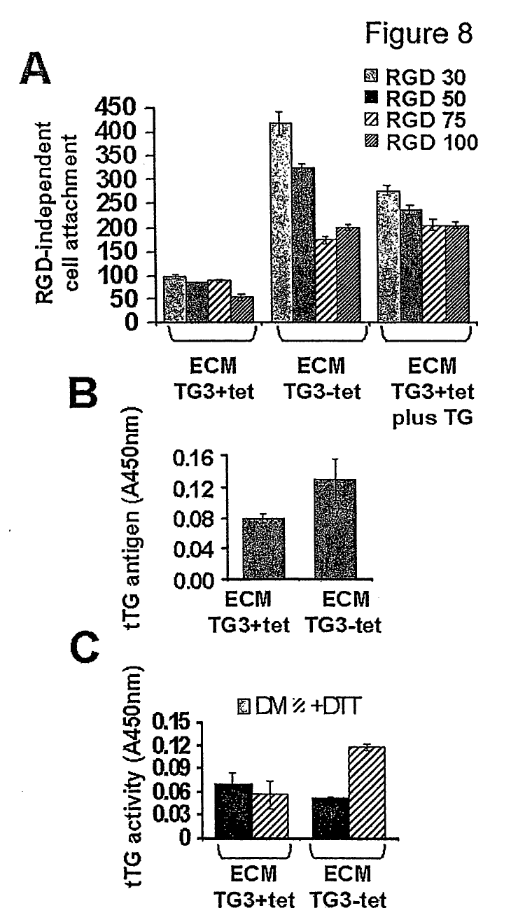

FIG. 8: Native FN matrix associated with cell-secreted tTG supports RGD-independent cell adhesion of Swiss 3T3 fibroblasts.

(A) Swiss 3T3 cells capable of inducible overexpression of tTG by withdrawal of tetracycline from the culture medium (transfected clone TG3) were grown on TCP, either in the absence of tetracycline in the medium, in order to induce the expression and secretion of tTG and thus deposit a ECM rich in tTG (ECM TG3-tet), or in the presence of tetracycline, to inhibit tTG overexpression and deposit a ECM and with background low levels of tTG (ECM TG3+tet). The cells were then removed by using 5 mM EDTA in PBS pH 7.4, and the remaining matrices were used to measure the attachment of untransfected Swiss 3T3 fibroblasts, pre-incubated with increasing concentrations of RGD peptide (30-100 μg/ml) (RGD 30-100). RGD-independent adhesion was also tested on the ECM from TG3+tet cells supplemented with purified tTG (20 μg/ml), which was immobilised as done on purified FN (ECM TG3+tet plus TG). Each point represents the mean number of attached cells (cell attachment)±SD, of triplicate wells of a representative experiment. (B) Relative levels of tTG found in the ECM of cells overexpressing tTG (TG3-tet) and not overexpressing tTG (TG3+tet). tTG was detected by an ELISA-type assay described earlier and in the Methods. Each value represents the mean±SD of six replicas from a representative experiment. (C) Cross-linking activity of tTG in the ECM of TG3+tet and TG3-tet fibroblasts. The activity of tTG was measured by assaying the level of incorporation of biotinylated cadaverine into fibronectin in culture medium DMEM in the absence of DTT (DM) or in the presence of 5mM DTT (DTT). Data are expressed as absorbances at 450 nm (tTG activity) as described in the Methods. Each value represents the mean absorbance±SD of three replicas from a representative experiment.

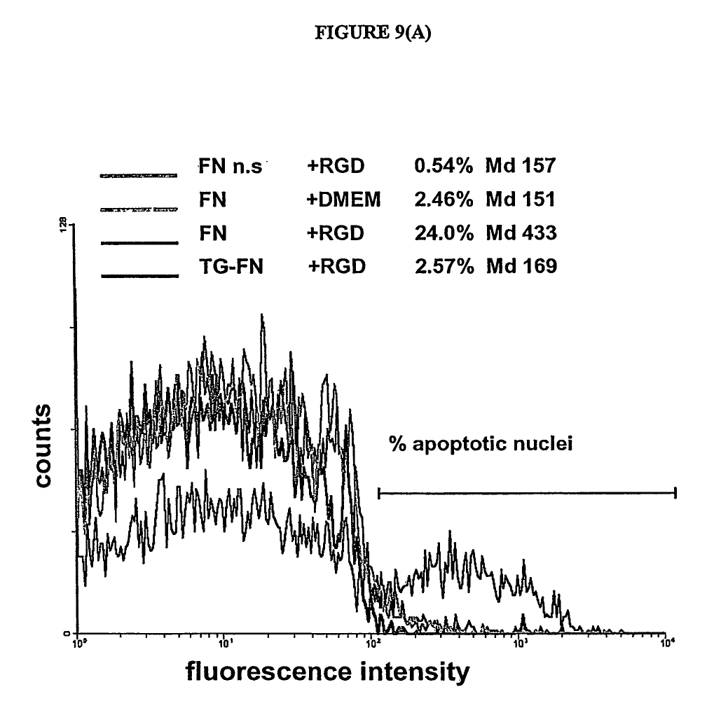

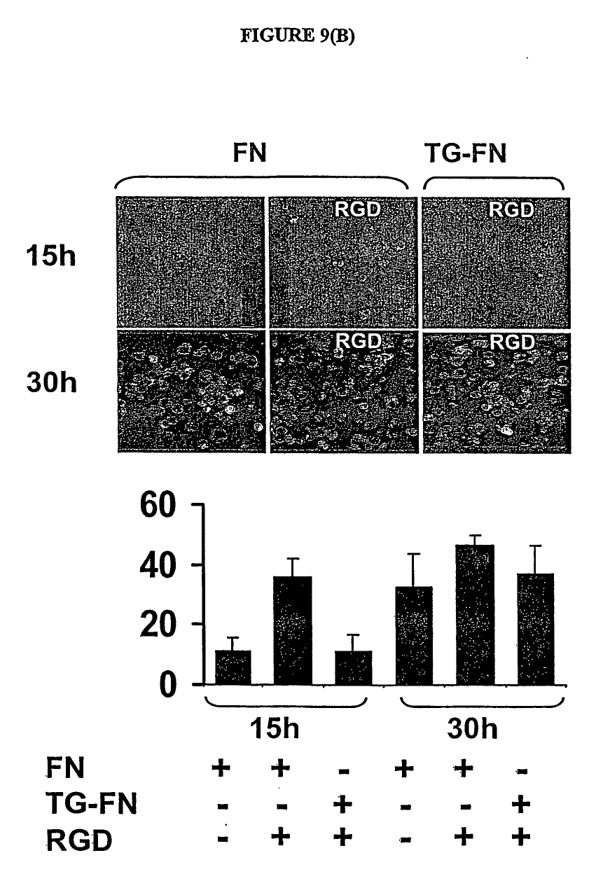

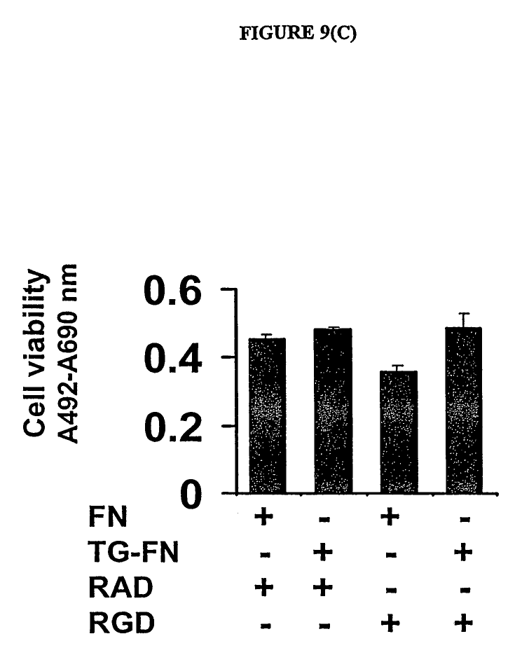

FIG. 9: Cell attachment to tTG-FN promotes cell survival of tTG-null dermal fibroblasts undergoing apoptosis by functional blocking of integrins with RGD peptides.

(A) Flow cytometric analysis of apoptosis in tTG-null mouse dermal fibroblasts (MDF-TG−/−). Cells were cultured in medium containing 100μ/ml of RGD peptide (RGD) or DMEM only (DMEM), on fibronectin (FN) or fibronectin with bound tTG (TG-FN) for 15 hours. Nuclear fragmentation of both adherent and detached cells was detected by fluorescent labelling of DNA strand breaks using the TUNEL method and then quantified by flow cytometry as described in the Methods. The bar denotes the positive, upper-channel region including fragmented nuclei (% apoptotic nuclei), set by the negative standard of cells undergoing anoikis and incubated with fluorescein dUTP in the absence of the enzyme TdT (n.s.,). Md, median fluorescence channel. (B) In situ analysis of nuclear fragmentation of MDF-TG−/−. Cells were treated with RGD peptide and allowed to attach on FN or TG-FN as described in A. After ˜15-hour and 30-hour growth, the fractions of cells in suspension were processed for in situ detection of DNA fragmentation, which was scored by confocal fluorescent microscopy as described in the Methods. Data are expressed as mean number of apoptotic cells/well±50. Total cells analysed ˜450 and ˜400, at 15 h and 30 h, respectively. (C) Cell viability assay of MDF-TG−/−. Cells were grown in medium containing 100 μg/ml of RGD peptide or control RAD peptide on FN or tTG-FN for ˜15 hours, as described above, after that cell viability was tested following incubation of cells with XTT. Each value represents the mean±SD of four replicates from a representative experiment.

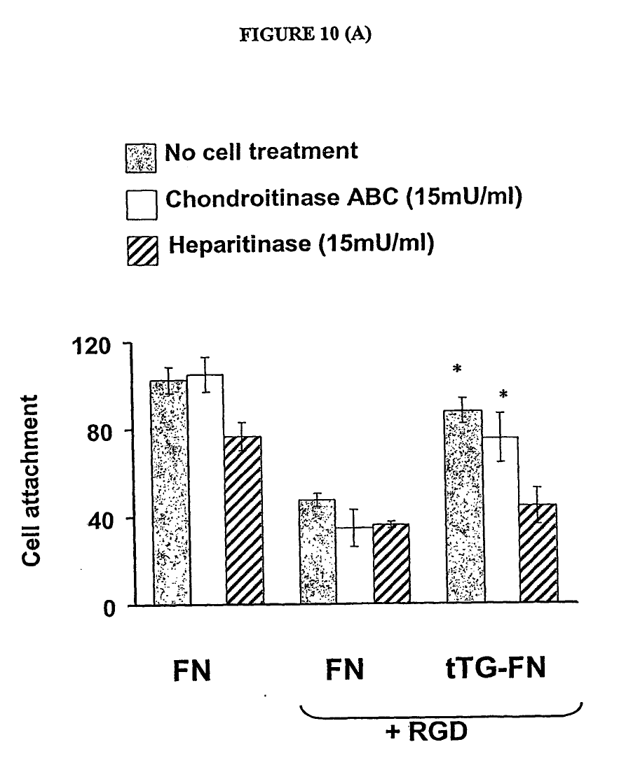

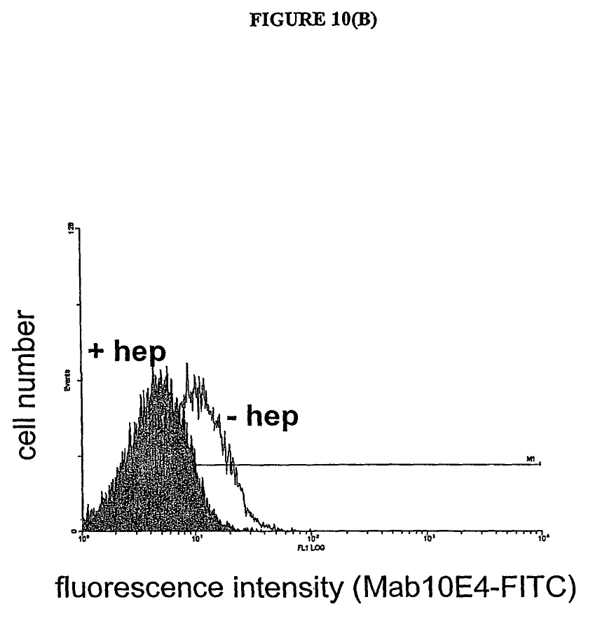

FIG. 10: RGB-independent cell adhesion to tTG-FN is dependent on cell-surface heparan sulfate

(A) Cell attachment was measured after seeding of BRIN-BD11 (clonal β-cells) onto fibronectin (FN) in the absence or presence of tissue transglutaminase (tTG-FN). Cells were pre-incubated with excess RGD peptide (+RGD) to inhibit the RGD-dependent integrin receptors (RAD peptide was used as control peptide). Each point represents the mean number of attached cells (cell attachment)±SD of triplicate wells and data are expressed as percentage of control values on FN. Differences between data sets were determined by the Student's t-test (two-tailed distribution, two-sample equal variance). The asterisk (*) indicates significant difference (p<0.05) between cell adhesion of cells on FN and tTG-FN.

(B) Human osteoblasts (HOB) grown in complete medium with foetal calf serum were detached from the substratum using PBS pH 7.4 containing 2 mM EDTA and then washed with DMEM without serum (0.5 ml). The cells in suspension were then either incubated with 50 mU/ml heparitinase I (Segakaku America) 1 h at 37° C. or with an equivalent volume of 50 mM Tris Cl. Heparitinase-treated and untreated cells were then washed in DMEM without serum and finally resuspended in 0.5 ml DMEM without serum. Cells were then incubated with a monoclonal antibody to Heparan Sulfate, which was FITC-conjugated (Mab 10E5-FITC, Seikagaku America) (8 mg/ml) for 2 h at 4° C. after that cells were washed and fixed in 0.5% formaldehyde-PBS and then examined by flow cytometry using a Beckman Coulter EPICS XL, following the manufacturer instructions. Data were stored using the SYSTEM II software and analysed using the WinMDI2.8 software.

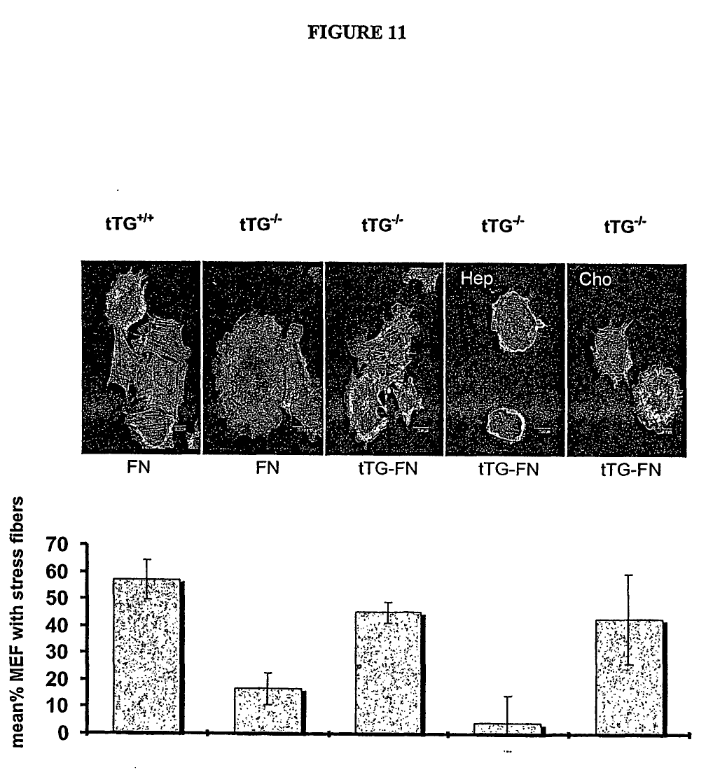

FIG. 11: Changes in tTG-FN-mediated actin assembly in heparitinase and chondroitinase-treated tTG^{-/-} MEFs

A cell suspension of wild type and tTG-deficient mouse embryonic fibroblasts (MEF-TG +/+and MEF-TG−/−, respectively) was prepared from cultures exponentially growing in complete medium with foetal calf serum. Cells were detached from substratum by using 0.25% (v/v) trypsin in 5 mM EDTA, collected into medium containing a ˜7% (v/v) final concentration of FCS, washed with medium without serum and re-suspended at the concentration of 8×10⁴ cells/ml in medium without serum. The suspension of MEF-TG−/− was pre-incubated with either 30 mU/ml heparitinase I (‘Hep’; 1000 mU/ml Segakaku America) or 30 mU/ml chondroitinase ABC protease free (‘Cho’; 1000 mU/ml), for 1 minute at 37° C. in medium without serum. As a control, both MEF-TG+/+ and MEF-TG−/− were also incubated with an equal volume of 50 mM TrisCl pH 7.0. The treated cells and the control cells were then seeded in 0.79 cm²-wells of chamber slides (2×10⁴/well) previously coated with FN and FN with immobilised tTG (tTG-FN) and allowed to adhere for ˜30 minutes. Cells were rinsed in PBS and fixed using 3.7% paraformaldehyde in PBS pH 7.4. After rinsing 3 times in PBS pH 7.4, cells were permeabilised in 0.1% TritonX-100 in PBS pH 7.4 and then washed in PBS pH 7.4. For detection of stress fibres, fixed and permeabilised cells were blocked in PBS buffer pH 7.4 supplemented with 5% (w/v) dry milk for 30 minutes at ambient temperature and then incubated with FITC-labelled phalloidin at the concentration of 15 μg/ml in blocking buffer for two hours at 37° C. in a humidified chamber. After further washes, coverslips were mounted with Vectashield containing propidium iodide (Vector Laboratories, Peterborough UK) and observed by confocal fluorescent microscopy using a Leica TCSNT confocal laser microscope system (Leica Lasertechnik, Heidelberg, Germany), equipped with an argon/krypton laser adjusted at 488 nm for fluorescein excitation. For quantification of the formed actin stress fibers consecutive scanning sections (˜2 μm) from the upper to the bottom attachment site of cells were overlaid as an extended focus image and imaged cells (from at least 5 random fields to consider at least 50 cells in control) were scored for stress fiber formation with the aid of the Leica TCSNT (version 1.5-451) image processing menu (Verderio et al. J Biol Chem 2003 278: 42604-42614).

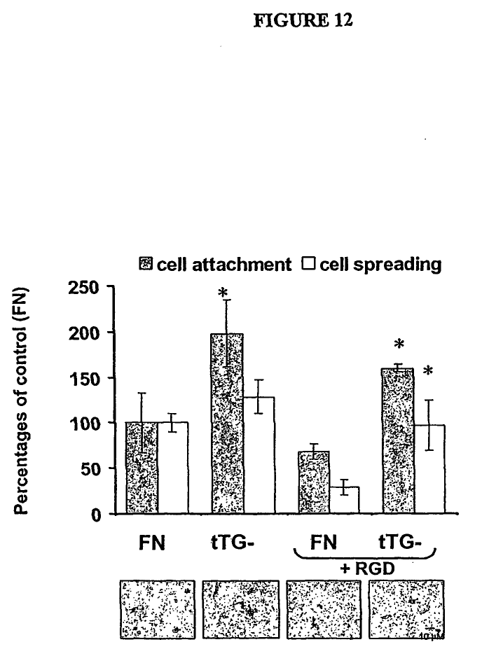

FIG. 12: Tissue transglutaminase bound to fibronectin supports RGD independent-cell adhesion of clonal β-cells

Cell attachment and cell spreading was measured after seeding of BRIN-BD11 (clonal β-cells) onto fibronectin (FN) in the absence or presence of tissue transglutaminase (tTG-FN). Cells were pre-incubated with excess RGD peptide (+RGD) to inhibit the RGD-dependent integrin receptors (RAD peptide was used as control peptide). Each point represents the mean number of attached cells (cell attachment) or the mean percentage of spread cells (cell spreading)±SD of triplicate wells and data are expressed as percentage of control values on FN. Differences between data sets were determined by the Student's t-test (two-tailed distribution, two-sample equal variance). The asterisk (*) indicates significant difference (p<0.05) between cell adhesion of cells on FN and tTG-FN.

### EXAMPLES

### Example A

A Novel RGD-Independent Cell Adhesion Pathway Mediated by Fibronectin-Bound Tissue Transglutaminase

Methods

Reagents and Antibodies

The following mouse monoclonal antibodies were utilised: function blocking anti-integrin β1 (JB1A) and anti-integrin α5 (PID6) (Chemicon), anti-vinculin (Sigma-Aldrich), anti-tTG (Cub74) (NeoMarkers), anti-tubulin (Sigma). Rabbit polyclonal anti-human fibronectin was obtained from Sigma; rabbit polyclonal anti-human phospho(Tyr 397)-FAK from Upstate Biotechnology. The tTG inhibitor R283 (Freund et al., 1994) was kindly synthesised by R Saint and I Coutts at Nottingham Trent University. Purified guinea pig liver tTG was either obtained by Sigma-Aldrich or purified in the laboratory according to Leblanc et al., 1999. Human plasma FN and FN proteolytic fragments, GTPγ-S, synthetic RGD specific peptides (GRGDTP) were all obtained from Sigma-Aldrich; control RAD peptide (GRADSP) was purchased from Calbiochem. Heparitinase (EC 4.2.2.8) was obtained by Sigma, and ChondroitinaseABC protease-free by Seikagaku. Unless otherwise indicated, all other chemical reagents were obtained from Sigma. The protein kinase Cα inhibitor GO6976 was obtained from Calbiochem.

Cell Lines

Primary human osteoblasts (HOB) were isolated from explants of trabecular bone dissected from femoral heads (DiSilvio, 1995, A novel application of two biomaterials for the delivery of growth hormone and its effects on osteoblasts. PhD Thesis, University of London). Cells were maintained in Dulbecco's modified Eagle's medium (DMEM) as previously described (Verderio et al. 2000). Swiss 3T3 albino fibroblasts were obtained from ATCC and maintained in DMEM supplemented with 10% FCS, 2 mM glutamine, and penicillin/streptomycin (100 U/ml and 100 μg/ml, respectively). Transfected Swiss 3T3 fibroblasts, displaying inducible expression of tTG (clone TG3), were cultured and induced. To induce tTG expression, cells were trypsinised and reseeded in medium without tetracycline as described by Verderio et al., (1998). Primary mouse dermal fibroblasts (MDFs) were isolated from the skin of tTG-deficient (MDF-TG−/−) and wild type (MDF-TG+/+) 9 months old mice and maintained as described by De Laurenzi and Melino, (2001).

Immobilisation of TG on FN and Detection of tTG and FN

The wells of a 96-well plate (0.32 cm²) were coated with human plasma FN (5 μg/ml) in 50 mM Tris-HCl, pH 7.4, 50 ∝λ/well, by incubation at 4° C. for approximately 15 h. For tTG immobilisation, the FN solution was removed, the wells were washed once in 50 mM Tris-HCl, pH 7.4 and then incubated with purified guinea pig liver tTG (20 μg/ml) in PBS pH 7.4 containing 2 mM EDTA pH 7.4, 100 μl/well. For control wells coated with tTG only, the tTG solution was added directly to tissue culture plastic (TCP). After one hour-incubation at 37° C., the tTG solution was removed and wells were washed once in 50 mM Tris-HCl, pH 7.4 and once in serum-free culture medium before cell seeding. The presence of tTG immobilised on FN was confirmed by an ELISA-type assay (Jones et al. 1997), using Cub 74 (1:5000). Absorbance values were read in an ELISA plate reader at 450 nm (Tecan Spectrafluor). Confirmation that the immobilised tTG was still active as a Ca²⁺-dependent ε-(γ-glutamyl)lysine crosslinker was gained by measuring tTG activity with a microtitre assay that is based on the incorporation of biotinylated cadaverine into FN in the presence of 5 mM CaCl₂ and 5 mM DTT (Jones et al. 1997) and compared with the activity of free tTG standard. To confirm that the cross-linking activity was Ca²⁺-dependent, 5 mM CaCl₂ was replaced with 5 mM EDTA pH 7.4. Absorbance values were read at 450 nm and data were expressed as absorbance value with Ca²⁺ in the reaction buffer minus background absorbance value with EDTA in the reaction buffer. Similarly to tTG, FN antigen was detected by using a modified ELISA based on a rabbit polyclonal anti-FN antibody (1/5000) followed by peroxidase-labelled anti-rabbit IgG (1/5000).

Immobilisation of TG on Amino-Terminal FN Fragments

The wells of a 96-well plate were coated with the 70 kd (42 μg/m), 45 kd (54 μg/ml), and 30 kd (54 μg/ml) proteolytic fragments of FN in 50 mM Tris-HCl, pH 7.4, by incubation at 4° C. for approximately 15 h. The concentrations of FN fragments were those optimal to saturate TCP, as measured by an ELISA-based assay with a polyclonal anti-FN antibody, and were in a 30-, 60- and 90-fold stoichiometric excess, respectively, of control FN (5 μg/ml). Immobilisation of tTG on the FN fragments was done essentially as described earlier on FN, and quantified by an ELISA-based assay using Cub7402 as described previously. All FN fragments reached saturating levels of bound tTG when incubated with a 20 μg/ml tTG solution as for complete FN.

Cell Adhesion Assay

To evaluate cell attachment and cell spreading exponentially growing cells were detached from substratum by trypsinisation (0.25% (v/v) trypsin in 5 mM EDTA), collected into medium containing a ˜7% (V/V) final concentration of FCS, washed twice with medium without serum and then plated (2×10⁴cells/well) onto FN-coated 96-well plates with and without further immobilisation of tTG. After a maximum of 20-minute incubation (to minimise the effect of any endogenous secreted proteins) at 37° C. in a 5% CO2 atmosphere, cells were fixed in 3.7% (w/v) paraformaldehyde in PBS pH 7.4, permeabilised in 0.1% (v/v) Triton X-100 and stained with May Grunwald and Giemsa stain (Jones et al 1997, Verderio et al. 2000). In some cases cells were pre-treated for 15 hours with 1 mM cycloheximide before plating to rule out any effects of endogenous secreted adhesion molecules. Digital images of three non-overlapping fields covering the central portion of each well was captured using a video digital camera (Olympus DP10) and examined using the Image Analysis programme Scion Image (National Institute of Health, USA). A total of at least nine images of separate fields per sample were examined for a total of at least 900 cells in the FN control. The number of cell particles in each field was measured by thresholding and particle analysis setting a minimum particle size of 50 pixels. Spread cells were analysed and quantified by “density slicing” and particle analysis.

Cytoskeletal Staining

Cytoskeletal rearrangements leading to cell spreading were assessed by staining the actin stress fibres using FITC-labelled phalloidin and revealing focal adhesions by staining for the marker protein vinculin. Cells were seeded in 0.79 cm²-wells of chamber slides (8×10⁴/well) previously coated with FN and TG-FN and allowed to adhere for ˜20 minutes. Cells were fixed using 3.7% paraformaldehyde in PBS pH 7.4 and permeabilised in 0.1% TritonX-100 in PBS pH 7.4. For detection of actin stress fibres, cells were then blocked in PBS buffer pH 7.4 supplemented with 5% (w/v) dry milk for 30 minutes at ambient temperature and then incubated with FITC-labelled phalloidin (20∝γ/∝γ) in blocking buffer for two hours at 37° C. For localisation of vinculin, cells were blocked in PBS buffer pH 7.4 containing 3% BSA and then incubated with mouse monoclonal anti-vinculin antibody (1:100) in blocking buffer for 15 h at +4° C. Bound antibody was revealed by incubation with rabbit anti-mouse-IgG -FITC (1:100) (Dako) in blocking buffer for 2 hours at room temperature. Coverslips were mounted with Vectashield containing propidium iodide (Vector Laboratories, Peterborough UK) and observed by confocal fluorescent microscopy using a Leica TCSNT confocal laser microscope system (Leica Lasertechnik, Heidelberg, Germany). For quantification of the formed actin stress fibres, consecutive scanning sections (˜2 μm) from the upper to the bottom attachment site of cells were overlayed as an extended focus image and imaged cells (from at least eight random fields, at least 100 cells in control) were scored for actin stress fibre formation with the aid of the Leica TCSNT (version 1.5-451) image processing menu.

Inhibition of Integrin-Mediated Cell Adhesion

Adhesion of cells to FN was decreased by pre-incubation with RGD synthetic peptide (50 μg/ml, ˜75 ∝M, 100 μg/ml ˜150 μM, or 200 μg/ml ˜300 μM) or function blocking anti-integrin antibodies 40 μg/ml anti-β1 integrin (JB1A), 30 μg/ml anti-α5 integrin (P1D6). Detached cells (as described above) were pre-incubated in suspension (2×10⁵cells/ml) in serum-free medium containing either RGD peptide or anti-integrins antibodies at 37° C. for 10 minutes in 5% CO₂ atmosphere, and then seeded in the presence of the peptides.

Pre-Treatment of Cells with C3 Exotranferase

Clostridium botulinum C3-exotransferase (Biomol Research Laboratories, PA) was loaded into cells using lipofectin reagent as a delivery vehicleC3 exotransferase (20 μg/ml) was incubated with lipofectamine (100 μg/ml) (Life Technologies, Paisley, Scotland) in DMEM at 22° C. for 1 hour. The C3 exotransferase/lipofectamine complex was then diluted further 10 times in DMEM and added to duplicate 18-hour old cell monolayers (˜80% confluent), in serum-free medium. After a 2-hour incubation the medium was removed and cells were allowed to recover for ˜30 minutes in serum-containing DMEM. Loaded cells were then seeded in 0.79 cm²-wells of chamber slides (45×10³ cells/well), pre-coated with either FN or tTG-FNand Actin stress fibres detected as described previously.

Analysis of FAK Tyrosine Phosphorylation

A cell suspension of 2×10⁵ cells/ml was seeded in 9.6 cm²-wells (6×10⁵ cells/well) pre-coated with FN, tTG-FN or heat-inactivated 3% BSA (w/v) in PBS pH 7.4. After 30-minute incubation at 37° C. in a 5% CO2 atmosphere, the supernatant medium was removed, and non-adhered cells collected by spinning at 270 g for 5 minutes and then lysed in reducing Laemmli sample buffer at 95° C. for 5 minutes. The layer of adhered cells was scraped and similarly lysed (200 μl/well). The lysates of adhered and non-adherent cells were pooled together, fractionated (¼ aliquot) on 8% SDS-PAGE and then western blotted. Blots were incubated with a polyclonal rabbit anti-human phospho(Tyr 397)-FAK (1/2000), for ˜15 hours at +4° C. followed by anti-rabbit IgG peroxidase-conjugate (1/2000) for 2 hours at 21° C. Bands were detected by chemi-luminescence (Amersham), following manufacturer instructions. For re-probing with a control antibody, blots were treated with stripping buffer [50 mM Tris.HCl pH 6.7, 100 mM βME, 2% SDS (w/v)] for 30 minutes at 50° C. and then incubated with a monoclonal anti-tubulin antibody (1/2000), followed by peroxidase-conjugate anti-mouse IgGs/(1/1000).

Quantification of Anoikis by Flow Cytometry and Fluorescence Microscopy and Measurement of Cell Viability

For fluorochrome labelling of DNA strand breaks, 6×10⁵ cells were seeded in duplicate in 9.6 cm²-wells pre-coated with FN or tTG-FN in the presence or absence of RGD peptide. After 15-hour incubation at 37° C. in a 5% CO2 atmosphere, all cells (adhered and non-adhered) were collected, washed twice in PBS pH 7.4, resuspended at the final concentration of 1.2×10⁷ cells/ml and fixed in suspension by addition of one volume of 4% (w/v) PFM in PBS, pH 7.4 for 1 hour at room temperature. The fixative was then removed by centrifugation (300 g for 10 min) and cells washed and permeabilised in 01% (v/v) TritonX-100 in 01%(w/v) sodium citrate buffer, for 2 min on ice (to minimise loss of fragmented DNA). Cells were labelled with terminal deoxynucleotidyl transferase (TdT) and FITC-dUTP, using a TUNEL kit according to the manufacturer (Roche). The fluorescence intensity measured by flow cytometry using a Beckman Coulter EPICS XL. Cells were collected and data stored using the SYSTEM II software and analysed using the WinMDI2.8 software. DNA fragmentation was also detected by in situ analysis of nuclei following TUNEL using a confocal fluorescent microscope (Leica). Cells found in suspension were labelled after fixing air-dried cells in triplicate on 0.79 cm²-wells of glass slides (˜5×10⁴ cells/well). For quantification, the Leica LCS software was used to acquire three random images per well for a total of 9 images per experimental sample using a fixed protocol (with constant PMT and section-depth setting). Data are expressed as mean number of apoptotic cells per well. Cell viability was assesses by a colorimetric (XTT) assay (Roche), following 4-hour incubation with the tetrazolium salt. Data are expressed as absorbance values at 492 nm after subtraction of values at 69 nm given by the unmetabolised salt.

Statistics

Differences between data sets (shown as mean±SD) were determined by the Student's t-test (two-tailed distribution, two-sample equal variance) statements made in the text which refer to “significant” or statistical differences indicate a significance level of P<0.05.

Results

Tissue Transglutaminase Bound to FN Supports RGD Independent-Cell Adhesion of Different Cell Types

Immunogold electron microscopy and immunofluorescence has previously demonstrated a close association of tTG with FN in dense clusters at the cell surface/pericellular matrix, which is consistent with the in vitro specific binding of guinea pig liver tTG with human plasma FN (Gaudry et al., 1999; Verderio et al., 1998; LeMosy et al., 1992). To investigate how this tTG-FN complex affects FN cell adhesion at the molecular level, we first bound purified guinea pig liver tTG to human plasma FN coated onto tissue culture plastic (TCP). EDTA was included in the binding reaction to rule out any tTG transamidating activity, since the complex between tTG and FN occurs even in the absence of calcium (Radek et al., 1993). Measurement of binding by an ELISA-type assay showed that FN, immobilised at the concentration of 5 μg/ml, bound a saturating amount of tTG when incubated with 20 μg/ml free tTG (FIG. 1A). Using this matrix model of immobilised FN with bound tTG the contribution of tTG to FN cell adhesion was examined by inhibiting integrin-mediated RGD-dependent cell adhesion with soluble RGD peptides. Human osteoblast-like cells (HOB) were selected as the initial cell model since they preferentially adhere on FN in vitro, demonstrate an enhanced spread morphology on biomaterials coated with tTG-FN (Heath et al., 2002) and are characterised by a simple and well defined pattern of integrin cell-surface receptors consisting mainly of RGD-binding β1 subunit paired with α1, α2, α3, α5 and αV subunits (Gronthos et al., 1997). When cell attachment and cell spreading were evaluated as indicators of cell adhesion (FIG. 1B), HOB cells appeared to attach to the tTG-FN matrix in a manner comparable to the matrix of FN in the absence of RGD peptide (FIG. 1B upper, RGD 0), although compared to FN alone the spreading of cells appeared to be enhanced on the tTG-FN matrix (FIG. 1B, lower). Negligible attachment was measured when cells were seeded on tissue culture plastic directly coated with tTG, confirming that cell adhesion was mediated by the tTG-FN matrix rather than tTG alone (data not shown). In the presence of both 50 and 100 μg/ml RGD peptides (RGD 50 and 100), attachment on FN was significantly reduced (typically to 30-50% of control values on FN) (B upper). The partial inhibition of cell adhesion is in agreement with the finding that the RGD-mediated mechanism does not completely account for the adhesion of osteoblasts (Gronthos et al., 1997). In contrast, cell attachment to tTG-FN was not significantly inhibited by addition of RGD peptides at both 50 and 100 μg/ml. Typically, cell attachment to tTG-FN matrix in the presence of 100 μg/ml RGD peptides was 85-95% of the cell attachment to FN control matrix (RGD 0). Only in the presence of higher concentrations of RGD peptide (200 μg/ml) (RGD 200), was cell attachment to the tTG-FN matrix significantly lower compared to control FN (RGD 0). However, at this high concentration the RGD peptide may in part cause some decrease in cell attachment independently from the RGD adhesion site, as shown by incubation with a control exapeptide containing the inactive RAD motif (inset B upper), which also led to reduction in cell attachment when used at 200 μg/ml (RAD 200) in comparison to untreated cells (RAD 0). Measurement of cell spreading showed that the spreading of HOB cells was significantly enhanced on tTG-FN compared to FN (FIG. 1B lower, RGD 0). Incubation of cells with excess RGD peptide, significantly reduced cell spreading on FN, typically to 10-50% of control value at 100 μg/ml RGD peptide, but in keeping with what was found for cell attachment, cell spreading was only partially reduced on tTG-FN at both 50 and 100 μg/ml RGD peptide (usually to 65-85% of control values on FN).

Rescue of cell adhesion in response to tTG-FN was not restricted to the osteoblast like cell line. Swiss3T3 fibroblasts displayed a comparable response. Attachment (FIG. 1C upper) and spreading (FIG. 1C lower) of Swiss 3T3 fibroblasts to FN was significantly decreased by incubation of cells with excess RGD peptide, in a more sensitive way (typically 30-40% of controls at 100 μg/ml RGD peptide) than in osteoblasts, however the inhibitory effect was restored to control levels by immobilisation of TG on FN when using inhibitory levels of RGD peptide at 50 and 100 μg/ml. Similarly, an epithelial-like cell line (ECV304) adhered more efficiently on tTG-FN than FN in the presence of excess RGD peptide (data not shown).

tTG Immobilisation on Amino-Terminal FN Fragments is not Sufficient to Mediate RGD-Independent Cell Adhesion

Having established the critical role of tTG immobilised on FN in stimulating RGD-independent cell adhesion we explored whether the N-terminal end of FN, which contains a putative tTG high affinity binding site, was sufficient to support this cell adhesion process (Jeong et al., 1995; Gaudry et al., 1999). Tissue culture plates were coated either with the 70 kd (matrix assembly, heparin and gelatin binding) or the 45 kd (gelatin-binding), or the 30 kd (first type I repeats of FN, matrix assembly, heparin binding) amino-terminal FN peptides and then tTG was allowed to bind to them. tTG detection by ELISA showed that incubation of the amino-terminal FN fragments with 20 μ/ml tTG resulted in saturating levels of tTG immobilised on all fragments (FIG. 2A), thus this concentration of tTG was utilised in all the adhesion experiments. HOB cells were allowed to adhere to the different tTG-FN fragments combinations for a maximum of 15-20 minutes, in order to minimise the secretion of endogenous ECM proteins. In the absence of integrin inhibition by RGD peptide, cells adhered efficiently on the 70 kd fragment (70 kd, −RGD) as both cell attachment and spreading values (FIG. 1B upper and lower, respectively) were ˜80% of values obtained on control FN, however cells adhered very poorly on the 45 kd and 30 kd fragments (45 kd and 30 kd, −RGD) since cell attachment and cell spreading (FIG. 2B upper and lower, respectively) were, in the order, ˜10% and 5% of control FN. In the presence of the RGD peptide, cell attachment and spreading values obtained with the 70 kd fragment were significantly inhibited by ˜40% (FIG. 2B upper and lower, respectively). This data is in agreement with previous findings that the amino-terminal of FN binds to the integrin α5β1 in cross-competition with the RGD peptide (Hocking et al. 1998). However, immobilisation of tTG on the 70 kd fragment did not induce RGD-independent cell attachment and cell spreading (FIG. 2B, TG-70 kd +RGD) as in response to tTG bound to the entire FN molecule (FIG. 1B TG-FN, +RGD), instead it led to what appeared to be further deterioration of the cell attachment and spreading inhibited by RGD peptide. Yet tTG did not enhance cell adhesion to the 70 kd fragment even in the absence of the RGD peptide (TG-70 kd, −RGD), in contrast to that observed with fill-length FN (TG-FN, −RGD) (FIG. 1B). tTG did not improve cell adhesion on the 45 kd and 30 kd FN fragments, which remained at negligible levels (FIG. 2B, TG-45 kd and TG-30 kd, +RGD and −RGD). These results demonstrate that adhesion of HOB cells to the amino-terminal 45 kd and 30 kd FN fragments, which contain putative tTG binding sites, is negligible regardless of the binding of tTG. They also demonstrate that cells can adhere to the 70 kd FN fragment but that tTG binding is not sufficient to sustain RGD-independent cell adhesion to the 70 kd peptide.

RGD-Independent Cell Adhesion to FN with Immobilised tTG Promotes Formation of Focal Adhesion Structures in Osteoblast-Like Cells

Formation of actin stress fibres in the presence of integrin-binding RGD peptide in response to tTG-FN was analysed by confocal laser scanning microscopy, utilizing FITC-phalloidin (FIG. 3A, actin fibres). RGD peptide reduced cell attachment to FN and in the residual adhered rounded cells, actin was not organised into stress fibres (FN+RGD) compared to cells adhered to FN (FN), which exhibited a flat polyglonal-like morphology and an extensive array of actin stress fibres. In contrast, cells seeded on tTG-FN had a spread morphology and showed the ability to organise actin in the presence of RGD peptide (TG-FN+RGD). However, the actin fibres formed on tTG-FN in the presence of RGD peptide were qualitatively different to those assembled in the control cells, being visually shorter and less well organised (TG-FN+RGD, see enlarged cell in inset). Interestingly, in the absence of the RGD peptide, actin stress fibres appeared to be more extensive and well-formed in response to tTG-FN (TG-FN, see enlarged cell in inset) compared to FN alone (FN), confirming the cell adhesion data that the immobilised tTG enhances cell spreading (see FIG. 1B and 2B). Visualisation of focal contacts by staining for the presence of the focal adhesion marker vinculin (FIG. 3A, vinculin), indicated the absence of punctate characteristic vinculin staining of FN-adhered cells (FN) in the RGD-treated cells adhered to FN (FN+RGD) but not in the RGD-treated cells adhered to tTG-FN (TG-FN+RGD). Relative measurement of the number of formed actin stress fibres (FIG. 3A, graph) confirmed these observations, that the presence of RGD peptide did not affect the formation of focal adhesions in cells plated on tTG-FN, although it significantly affected the quality of the actin reorganisation, as measured by fluorescence microscopy.

The RGD-independent formation of the actin cytoskeleton in HOB cells in response to tTG-FN was further explored by inhibiting integrins ®1 and <5 with anti-functional anti-integrins monoclonal antibodies directed either towards the RGD-interacting site on the β1 subunit (JB1A) or the site on the α5 subunit interacting with the adjacent PHSRN synergy sequence (PID6). Treatment of cells with either JB1A (FIG. 3B FN/anti-β1) and, to a lesser extent PID6 (FIG. 3B FN/anti-α5), led to a large decrease of actin stress fibres, when compared to cells incubated with control mouse non-specific IgG (FIG. 3B FN/IgG). In contrast, when seeded on tTG-FN, a large fraction of cells incubated with the anti-integrin antibodies appeared to maintain an organised network of actin stress fibres (tTG-FN/anti-β81and tTG-FN/anti-α5), although less elaborated and dense than in control cells treated with mouse IgGs (FN/IgG). Relative measurement of the number of formed actin stress fibres (FIG. 3B, graph) statistically confirmed these observations and showed that the anti-β1 integrin antibody had a greater inhibitory effect than the anti-α5 integrin antibody, in agreement with the observation that integrin β1 is the predominant integrin receptor subfamily in osteoblasts like cells (Gronthos et al., 1997), and that the antibody-inhibition was largely reduced when cells were plated on tTG-FN. Since the assembly of focal adhesions and stress fibres is normally regulated by GTPase RhoA, we next determined whether adhesion of cells to the tTG-FN matrix in the presence of RGD inhibition stimulated Rho activation. RhoA activity was blocked by pretreatment of cells with the botulinum toxin C3 exotransferase, which was delivered in the form of a lipofectin complex. Relative measurements of actin stress fibres (FIG. 3C) showed that inhibition of RhoA activity by pre-treatment of cells with C3 exotransferase (C3) almost completely blocked assembly of actin stress fibres in response to both FN (FN) and FN with immobilised tTG (TG-FN). Inactivation of RhoA also significantly inhibited the RGD-independent formation of actin stress fibres mediated by tTG-FN (FIG. 3C, TG-FN+RGD). This finding confirms that activation of GTPase RhoA is necessary for the adhesion of cells in response to tTG-FN.

Tissue TG Cross-Linking Activity is not Required to Support RGD-Independent Cell Adhesion

FIG. 4A shows that, the activity of tTG once sequestered by FN binding (TG-FN) is negligible, when the assay is performed in cell culture medium (DM) containing an activating concentration of calcium (˜1.9 mM), thus simulating the experimental conditions of the cell adhesion assay. The transamidating activity does not change upon addition of up to 5 mM (DM-Ca²⁺) to the culture medium, , but it is significantly boosted (approx 6 fold) by the addition of reducing agent (DM-DTT). Since a similar increase in transamidating activity was observed when tTG was pre-incubated with DTT prior to immobilisation on FN, (to rule out any effects of DTT on FN, data not shown), this finding suggests that under the conditions used FN-bound tTG is not in an active state in the presence of cell culture medium, unless its Cys residues, and in particular the active-site Cys 277, are kept in a reduced state. The transamidation-independent role played by tTG in the RGD-independent cell adhesion process was further confirmed by utilizing a 2-[(2-oxopropyl)thio]imidazolium derivative, which is a known active-site directed tTG inhibitor (named R283) (Freund et al., 1994). HOB cells were incubated with R283 (100 μM) and plated on a tTG-FN matrix pre-treated with R283, in the absence or presence of RGD peptide. Under these conditions using the same batch of inhibitor, the transamidating activity of the immobilised tTG, is completely blocked by the inhibitor (R283) (FIG. 4C). In addition, the low tTG activity found at the HOB cell-surface (Verderio et al. 2000; Heath et al., 2001) is also inhibited to negligible values by R283 treatment (data not shown). Cell adhesion on FN bound to tTG enzymatically inactivated by R283 was not significantly different to cell adhesion on FN bound to tTG in the absence of R283, with or without pre-treatment of cells with RGD peptide and it was typically twice cell adhesion on FN in the presence of RGD peptide (FIG. 4B; cell attachment, upper and cell spreading, lower). Consistent with this finding, focal adhesions still showed positive staining for vinculin in cells plated on FN bound to inactive tTG in the presence of RGD peptide, in a comparable way to cells plated on FN bound to active tTG (data not shown). Taken together these data clearly indicate that FN-bound tTG does not require its transamidating activity to promote RGD-independent cell adhesion.

Evidence for the Importance of tTG and its Calcium-Induced Conformation in Mediating the RGD-Independent Cell Adhesion

To assess the importance of FN-associated tTG in cell-surface recognition, FN-bound tTG was blocked by using the monoclonal anti-tTG antibody Cub 74, in conditions that fully preserved the availability of FN in this complex. This was demonstrated by the unchanged recognition of FN by anti-FN polyclonal antibody after treatment of tTG-FN with either Cub74 or control IgG, as shown by ELISA (FIG. 5B). Obstruction of tTG by Cub74 completely inhibited the RGD-independent cell attachment and spreading mediated by the tTG-FN matrix in the presence of control IgG, since cell attachment on tTG-FN blocked by Cub 74 was not significantly different to cell attachment on FN in the presence of RGD peptide (FIG. 5A; cell attachment, upper and cell spreading, lower). This finding suggests a direct role for FN-associated tTG in the RGD-independent binding to cells. We next investigated the importance of the conformation of tTG in the RGD-independent cell adhesion induced by tTG-FN. The tTG three-dimensional structure indicates that the molecule assumes predominantly two conformations, depending on whether it is bound to GTP/GDP or calcium (Liu et al., 2002). In the extracellular environment, given the high calcium/GTP ratio, tTG is likely to assume the calcium-induced open structure. Incubation of FN-bound tTG with the non-hydrolysable GTP-γS (1 mM), partially but significantly reduced the RGD-independent cell attachment and spreading mediated by tTG-FN (FIG. 5C; cell attachment, upper and cell spreading, lower). Incubation of tTG with GTP-γS did not significantly alter the level of tTG immobilised on FN, as shown by ELISA using Cub 74 (FIG. 5D). We found that under similar conditions, GTP-γS also acts as transamidating inhibitor of FN-bound tTG in culture medium DMEM supplemented with reducing agent (data not shown). Our results suggest that the RGD-independent cell adhesion induced by tTG-FN is dependent on the calcium-mediated tertiary structure of tTG.

Role of Heparan Sulfate proteoglycans in RGD-Independent Cell Adhesion and Signalling Via tTG-FN

To explore the possibility that cell adhesion to tTG-FN may be mediated by a cell-surface HSPG (Woods and Couchman, 2001; Jeong et al., 2001), HOB cells were treated with glysosaminoglycan-degrading enzymes. Removal of cell-surface heparan sulfate chains by pre-treatment of cells with heparitinase at 15 mU/ml, led to reduced cell attachment and spreading on FN (typically ˜70% of control values) for attachment but completely abolished RGD-independent cell attachment and spreading on tTG-FN (FIG. 6A). In contrast, equal concentrations of protease-free chondroitinase ABC had no significant affect on cell adhesion to FN and did not significantly alter RGD-independent cell spreading on tTG-FN, but only marginally affected the level of cell attachment (FIG. 6A). These results implicate cell-surface proteoglycans in RGD-independent cell adhesion to tTG-FN and suggest that heparan sulfate proteoglycans but not chondroitin sulfate proteoglycans are the main signalling receptors for tTG-FN. Of the heparan sulfate proteoglycans so far characterised, Syndecan-4 is the only HSPG that is a widespread component of focal adhesion (Woods and Couchman 2001). Downstream signalling of this HSPG specifically involves protein kinase Cα (PKCα). In order to characterise the HSPG responsible for tTG-FN mediated cell adhesion, HOB cells were treated with the PKCα inhibitor GO6976 prior to and during the cell adhesion experiments on FN and tTG-FN in the presence of the RGD peptide or the RAD control peptide as outlined previously. Cells seeded on FN in the presence of the control peptide RAD indicated significant (˜30%) inhibition of both attachment and spreading in the presence of the inhibitor (FIG. 6B), which is comparable to the inhibition of attachment shown after treatment of cells with heparatinase (FIG. 6A). The inhibitor was also found to significantly augment the inhibition of attachment and spreading mediated by the RGD peptide. However in the cells seeded on the tTG-FN complex, the PKCa inhibitor gave rise to significant inhibition of attachment (over 90%) and spreading (˜85%) mediated by the tTG-FN complex in the presence of the RGD peptide suggesting that the HSPG responsible for binding to the tTG-FN complex is Syndecan4.

RGD-Independent Adhesion in Response to tTG-FN Enhances Tyrosine Phosphorylation of FAK

We next examined whether cell attachment to tTG-FN resulted in tyrosine phosphorylation of FAK. The levels of tyrosine phosphorylated FAK was revealed in total cell lysates by western blotting using anti-PTyr397 antibody, and data normalised by reference to the control tubulin protein (FIG. 7). Tyrosine phosphorylation of FAK in cells plated on FN and treated with RGD peptide was decreased to ˜25% the level found in control cells plated on FN and pre-incubated with control RAD peptide. In response to tTG-FN instead, the level of FAK phosphorylation in the presence of RGD peptide was found to be ˜60% the level of both control cells on FN or tTG-FN. This data shows that rescue of cell adhesion by tTG-FN following blocking of integrins via RGD peptide, enhances tyrosine phosphorylation of FAK and as such implies that FAK is one of the mediators of tTG-FN.

RGD-Independent Cell Adhesion by a Physiological Matrix of Cell-Assembled FN and Cell-Secreted tTG

To confirm the existence of the tTG-mediated RGD-independent cell adhesion process in a matrix system that more closely relates to the ECM in vivo, a matrix of cell-assembled FN with bound cell-secreted tTG was obtained from a long-term culture of a fibroblast cell line (Swiss 3T3-TG3), capable of inducible expression of tTG (Verderio et al., 1998). Depending on the presence or absence of tetracycline in the medium, these cells can deposit a FN-rich ECM which in the induced cells (ECM TG3−tet) contains significantly increased levels of transfected tTG compared to the non induced cells (ECM TG3+tet) (Verderio et al., 1998), (FIG. 8B). Following matrix deposition, the inducible cells were removed from both matrices, using PBS with 5 mM EDTA, and in their place wild-type Swiss 3T3 fibroblasts were seeded and allowed to adhere in the presence of increasing concentrations of RGD peptide (30-100 μg/ml). The FN-rich ECM with increased amount of cell-secreted tTG (ECM TG3−tet) supported significantly higher RGD-independent cell attachment of fibroblasts at each RGD peptide concentration, compared to the ECM with background levels of tTG (ECM TG3+tet) (FIG. 8A). RGD-independent cell attachment on the low-tTG ECM could be significantly increased by immobilisation of purified tTG on this matrix (ECM TG3+tet plus tTG) (FIG. 8A). However, at low levels of RGD peptide, RGD-independent cell attachment was still more effective on the ECM containing cell-secreted tTG (ECM TG3-tet) than on the ECM containing increased tTG by exogenous addition (ECM tTG3+tet plus tTG). This is despite higher levels of tTG detected by ELISA following addition of exogenous TG (data not shown), suggesting that cell-secreted tTG is more able to present itself to the cell-surface, probably as a consequence of the close association with FN during its externalisation from cells (Verderio et al. 1999; Gaudry et al., 1999). Interestingly, as seen for purified tTG immobilised on FN (FIG. 4A), the ECM deposited by cells with increased levels of tTG (ECM-TG3−tet), showed no significant difference in tTG enzymatic activity above background (ECM-TG3+tet) in culture medium (DAM, unless in the presence of reducing agent (DTT) (FIG. 8C), consistent with the idea that tTG gradually looses its transamidating activity once sequestered in the oxidising extracellular environment.

FN-Bound tTG Rescues Primary Dermal Fibroblasts from Anoikis

A potential physiological function for the TG-mediated RGD independent cell adhesion and mediated intracellular survival signalling, is the protection from apoptosis triggered by inhibition of the RGD-dependent integrin function (anoikis) following tissue injury when changes in the composition of the ECM and its molecular structure frequently occurs. To examine the specific role of tTG-FN in anoikis we utilised non-confluent cultures of mouse dermal fibroblasts devoid of tTG (MDF-TG−/−) plated on FN or EN with immobilised tTG, and incubated with RGD peptide under serum-free conditions. After ˜15-hour incubation, the majority of the RGD-treated cells plated on FN were detached and displayed morphological signs of apoptosis. To identify and quantify apoptotic cells, the extent of endonucleolysis was measured by fluorescent labelling of DNA strand breaks using TUNEL. Label intensity of the apoptotic cells was quantified by flow cytometry (FIG. 9A) and apoptosis was judged on the basis of the increased mean intensity of fluorescence compared to identically grown control cells subjected to TUNEL but without the fluorescent label (FIG. 9, n.s, red histogram). After ˜15-hour incubation with the RGD peptide, ˜24% of the total fibroblasts (adhered and not adhered) grown on FN were apoptotic (FN+RGD, black histogram) while only ˜2.5% of fibroblasts grown on tTG-FN and exposed to the RGD peptide were apoptotic (TG-FN+RGD, blue histogram), similarly to control fibroblasts grown on FN in the absence of RGD peptide (FN+DMEM, green histogram). The result of the flow cytometric analysis was corroborated by in situ fluorescent labelling of nuclei undertaken on the detached fraction of cells in suspension (FIG. 9B), DNA fragmentation, which was visualised and scored by confocal microscopy. At 15 h a significantly lower number of apoptotic cells was found in the cell culture fluid of those cells incubated on tTG-FN when compared to those incubated on FN alone. The number of apoptotic cells, coming from cells incubated on the tTG-FN matrix, were found to be comparable to those cells incubated on FN without the addition of the RGD peptide. Unlike epithelial and endothelial cells, which are very susceptible to anoikis, it is anticipated that a longer incubation period with RGD peptide may be required to increase apoptosis in fibroblasts, as reported (Hadden et al., 2000). However, after 30-hour incubation in the absence of serum, nuclear fragmentation appeared to increase not only in cells grown with RGD peptide but also in cells grown on FN without RGD peptide, thus limiting our investigations to ˜15-hour time-period (FIG. 9B). We next assayed the viability of MDF-TG−/− in response to tTG-FN (FIG. 9C). Following blocking of integrins by RGD peptide for ˜15 hours, cells exhibited different survival characteristics on FN and tTG-FN. Cell viability on FN was decreased by around 20% after incubation with RGD peptide compared to control RAD peptide, consistent with the level of total apoptotic cell death detected in the same conditions (FIG. 9A). In contrast, cell viability on tTG-FN was not substantially altered by incubation with RGD peptide and it was found to be ˜25% higher than found on FN with RGD peptide, confirming that the attachment to tTG-FN mediates RGD-independent cell survival.

Discussion

Cell adhesion to the ECM is recognised as an important regulator of apoptosis, and subtle changes in the ECM complexity/tissue architecture may be crucial for the suppression or activation of the apoptotic machinery (Aplin et al., 1999; Frisch and Screaton, 2001). Such a process occurs during tissue injury when the composition of the ECM and its molecular structure are altered in several significant ways (Davis et al., 2000). The molecular structure of FN, an important matrix and plasma protein, is thought to greatly affect cell shape, migration, signalling and proliferation (Sechler and Schewarzbauer, 1998; Sechler et al., 2000; Hocking et al., 2000), it is therefore conceivable that modulation of the FN matrix can also regulate adhesion related apoptosis. In this context the impact of tissue transglutaminase, a well characterised FN-associating protein and modulator of the FN matrix (Jeong et al., 1995; Achyuthan et al., 1995; Martinez et al., 1994), on FN-mediated cell survival has never been investigated.

Whereas previous studies have analysed the roles of tTG by modulating its expression (Gentile et al., 1992; Verderio et al., 1998; Melino et al., 1997; Ritter and Davies, 1998), in the present study we have developed a model that allows us to characterise cellular responses to a tTG-rich fibronectin matrix, thus mimicking physio/pathological conditions in vivo. Support for our model comes from a number of findings indicating that tTG is not only externalised under normal physiological conditions but it is also upregulated and exported to the extracellular matrix in response to tissue trauma following cellular damage, inflammation or cell stress (Upchurch et al., 1987; Johnson et al., 1999; Haroon et al., 1999). Once externalised, tTG accumulates and associates with the extracellular matrix by virtue of its high affinity for FN, either by direct binding to FN fibrils or by binding to plasma FN which is then deposited in the damaged area (Lorand et al., 1988). Given the specific association of tTG with FN (Radek et al., 1993; Gaudry et al., 1999) and tTG involvement with cell adhesion (Gentile et al., 1992; Jones et al., 1997; Verderio et al., 1998; Belkin et al., 2001), it is conceivable that, upon binding, tTG and FN may modulate each others function and form a matrix complex with distinct adhesive roles. It is known for example that binding of FN to tTG forms a complex that protects tTG from degradation by matrix metalloproteinases (Belkin et al., 2001). Hence a complex of tTG and FN may provide a mechanism to ensure adhesion-mediated cell survival, in situations of cell wounding or cell stress, where the increased expression of matrix-degrading metalloproteinases triggered by the inflammatory response, would lead to increased degradation of the ECM, (Kapila et al., 1999; Haden and Henke, 2000).

To test this hypothesis, we initially examined the function of the tTG-FN complex in cell-matrix interactions whereby we inhibited the “classical” adhesion-mediated survival pathway dependent on the interaction of the RGD fibronectin cell binding site with α5β1 integrins. A human osteoblast like cell line served as the initial cell model since osteoblasts secrete both FN and tTG, are subject to continuous matrix remodelling processes during their differentiation (Heath et al., 2001), are characterised by a well defined and simple pattern of integrin cell surface receptors, furthermore they make use of RGD-independent pathways in the attachment to the ECM (Gronthos, 1997). Using this cell model we demonstrate that loss of cell-matrix interaction by inhibition of RGD-dependent integrin function using competitive peptides, can be largely re-established upon seeding of cells on either FN with immobilised tTG or a more physiological matrix of cell-assembled FN containing cell-secreted tTG. This latter form of tTG-FN matrix is thought to be the most important form present in vivo as in tissues FN is present as an insoluble fibrillar matrix to which tTG is closely associated (Verderio et al. 1998; Gaudry et al., 1999). Restoration of cell adhesion following inhibition of the RGD-dependent pathway was not only confined to the osteoblast-like cells but was also found in mouse Swiss 3T3 fibroblasts, , and in the epithelial-like cells ECV-304, suggesting that many cell types can use the RGD-independent cell-adhesion pathway mediated by tTG-FN.

Our data demonstrate that a matrix of tTG-FN stimulates the formation of focal adhesion complexes independently from the integrin RGD-binding site, following its inhibition by either RGD competitive peptide or function blocking anti-integrin monoclonal antibodies. The actin stress fibres formed appeared less complex than normal, nevertheless the observed preservation of cytoarchitecture was sufficient to support the formation of focal contacts and to give the cells a spread morphology. This supports the idea that tTG can act as a positive modulator of matrix FN, by inducing the formation of distinct, RGD-independent focal adhesion structures. This process depends on the activity of the small GTPase RhoA and induces FAK tyrosine phosphorylation. FAK has been shown to play a key role in integrin-stimulated signalling, however it has also been shown that integrin activation is not needed for FAK activation and an increasing number of non-integrin mediated stimuli have been described which enhance FAK tyrosine phosphorylation levels through mechanisms generally dependent on actin polymerisation (reviewed in Schlaepfer et al., 1999).

Our data clearly indicate that cell adhesion to tTG-FN is not linked to modification of FN by calcium-dependent transamidation. This finding is consistent with recent observations indicating a transamidating-independent role for tTG in cell-matrix interactions (Akimov et al., 2000; Balklava et al., 2002). This hypothesis finds further support from the finding presented here that when tTG is complexed with FN it becomes catalytically inactive unless kept in a reduced state. Indeed, previous work has described that abundant tTG found in lung matrix requires activation by reducing agents (Cocuzzi et al., 1986) and that tTG sequestration by FN leads to downregulation of enzymatic activity on large-size protein substrates (Lorand et al., 1988). In contrast, in situ tTG activity assay demonstrated with small-size fluorescent primary amine substrate has clearly shown that tTG is still in a catalytically active state while still present at the cell surface (Layemi et al., 1997; Verderio et al., 1998). It is also possible that tTG may modify FN by its intrinsic protein disulfide isomerase (PDI) activity, which has been recently ascribed to it (Hasegawa and Saito, 2002). However, the finding that GTP-binding affects the adhesion function of FN-bound tTG but it does not seem to affect the PDI activity of tTG, renders this possibility less likely (Hasegawa and Saito, 2002).

The outside-in signalling induced by the RGD-independent cell adhesion to tTG-FN appears to result by direct interaction of tTG with the cell surface, since the blocking of tTG accessibility by a monoclonal antibody greatly reduces this process. In addition, the calcium-mediated tertiary structure of tTG is required, suggesting that crucial cell binding sites might be exposed when tTG assumes the calcium-induced open conformation. However, the simple binding of tTG to either TCP or the gelatin binding domain of FN (45 kd peptide), which contains the tTG binding site (Radek et al., 1993), does not enhance cell adhesion, which is indeed equally negligible on the 45 kd peptide regardless of the presence of tTG. This finding is in contrast to what was reported when using tTG-overexpressing transfected cells (Akimov et al., 2000). It was also found that tTG binding to the 70 Kd amino-terminal fragment of FN, which can support cell adhesion and includes the tTG binding site, is not sufficient to sustain tTG-mediated RGD-independent cell adhesion. This leads us to conclude that this cell adhesion process is both tTG-and FN-dependent, with the amino-terminal part of FN required to support TG binding, while the carboxy-terminal part of FN and/or cryptic epitopes outside FNIII9-10 are essential to sustain the RGD-independent pathway. Our data also suggest that the RGD-independent cell adhesion to tTG-FN is integrin-independent or, alternatively, the integrin must be in partnership with some other receptor(s). In fact tTG-mediated focal adhesions still form in the presence of function blocking anti-integrin antibodies α5 and β1, which are known to cause conformational inactivation of the receptor, thus disabling target integrins to transduce outside in signalling (Humphries, 2000). The possibility that tTG-FN may mediate RGD-independent cell adhesion through the α4β1-induced RGD-independent pathway is also unlikely since low or negligible levels of α4 subunit are generally expressed in osteoblast-like cells (Gronthos, 1997). Moreover matrix binding to α4β1 does not generate actin stress fibres (Sechler et al., 2000). Although tTG has been found to associate with integrins (Akimov et al., 2000; Takahashi et al., 2000), our data suggest that it is more likely that the cell function induced by tTG-FN results from the binding to non integrin receptors.

. Treatment of HOB cells with heparitinase, greatly diminished the RGD-independent adhesion in response to tTG-FN. However treatment of cells with chondroitinase ABC did not affect the process suggesting that cell-surface HSPGs may mediate RGD-independent cell adhesion to tTG-FN. The C-terminal heparin-binding domain of FN (HepII) is responsible for the synergistic interaction of EN with cell-surface heparan sulfate and integrins (Lyon et al., 2000) and this interaction is essential for optimal cell adhesion and critical for sustained cell survival (Jeong et al., 2001). Therefore, the association of tTG with FN could induce RGD-independent cell adhesion by reinforcing HSPGs-mediated cell adhesion, through a mechanism involving both the binding of tTG to cell-surface HSPGs and increased exposure of the C-terminal heparin-binding domain of FN, which critically depends on FN structure (Sharma et al., 1999). The increased level of cell spreading observed in both osteoblasts and Swiss 3T3 fibroblasts in the absence of integrin inhibition in early cell adhesion to tTG-FN, suggests that cell-surface heparan-sulfate proteoglycans are not fully occupied when cells are normally seeded on a FN matrix. Downstream signalling from syndecan4 HSPG, the only HSPG that is a widespread component of focal adhesion (Woods and Couchman, 2001) specifically results in hyperactivation of protein kinase C (PKC)< and involves activation of both RhoA, and FAK ( Jeong et al., 2001; Wilcox et al., 2002). Our results suggest that tTG-FN mediated cell adhesion seems to require PKC< activity since the inhibitor GO6976, which at-low concentration has high specificity for PKCα(Gschwendt et al., 1996), also blocks RGD-independent cell adhesion to tTG-FN.

From a physiological context, the present study has shown that the function of matrix-bound tTG is linked to the adhesion mediated cell survival kinase activity of FAK. The mechanism leading to regulation of cell survival of anchorage-dependent cells by FAK has been extensively investigated and shows that many pathways are involved including induction of anti-apoptotic proteins such as the caspase inhibitors of the IAP family (inhibitor of apoptosis) (Sonoda et al., 2000). In preliminary investigations using a Raf-1 null 3T3 like immortalised fibroblast cell line (Mikula et al., 2001) we can demonstrate that the RGD independent cell adhesion pathway mediated by tTG-FN is not functional in this cell line but is functional in the corresponding wild type cells (data not shown). This suggests that the Raf-1 protein whose function is thought to be anti apoptotic (Mikula et al., 2001; Huser et al., 2001) may be a key component in the signalling pathway mediated by tTG-FN.

The observation that tTG-FN can rescue tTG-null primary dermal fibroblasts from detachment-induced apoptosis with maintenance of cell viability also demonstrates that the survival role of tTG is essentially extracellular and not intracellular as recently suggested by Antonyak et aL, (2001). These authors showed that protection by retinoic acid against apoptosis induced by synthetic retinoid analogues could be correlated to the expression of intracellular tTG in HL60 cells. It is now accepted that the RGD-mediated cell adhesion is not sufficient in isolation to maintain cell survival. For sustained survival cells need to interact with “complex” ECMs via integrin and non-integrin receptors, such as cell-surface proteoglycans (Jeong et al., 2001). Interestingly increased expression of Syndecan4 at sites of tissue injury is now thought to be an important factor in the wound response (Gallo et al., 1996). The work presented here indicates that binding of tTG to FN represents one additional survival signal by inducing HSPGs-mediated cell adhesion when RGD-dependent cell adhesion is reduced or blocked. Such a scenario may be envisaged whereby tTG, released in response to damage or stress, when bound to FN, either directly to matrix FN or via binding to plasma FN which is deposited at the site of injury, induces activation of survival signals via FAK in an RGD-independent manner.

While our investigations show that FN-bound tTG may act as an anti-apoptotic agent in anchorage-dependent cells, several studies have demonstrated a cell death function of tTG when expressed intracellularly. Work from this laboratory and others has shown that tTG is capable of causing massive intracellular protein cross-linking when calcium homeostasis is disturbed, resulting in a death mechanism that is both Bc1-2 and caspase-independent (Johnson et al., 1998; Griffin and Verderio, 2000). A recent report also suggests that upregulation of tTG in neuroblastoma cells sensitises these cells to cell death (Piacentini et al., 2002). In this context tTG-mediated cell death may therefore only represent the fmal scenario, and that the initial and possibly most important role of tTG, is to promote adhesion mediated cell survival in combination with its high affinity binding partner FN.

### Example B

RGD-Independent Cell Adhesion to tTG-FN is Dependent on Cell-Surface Heparan Sufate

Methods

Cell Attachment

Cell attachment was measured using the cell adhesion assay described in Example A (see above).

Detection of Cell Surface HSPG by Flow Cytometry using Mab 10E4 (Seikagaku America)

Human osteoblasts (HOB) (2×10⁶) grown in complete medium with foetal calf serum were detached from the substratum using PBS pH 7.4 containing 2 mM EDTA and then washed with DMEM without serum (0.5 ml).

The cells in suspension were then either incubated with 50 mU/ml heparitinase I (Segakaku America) 1 h at 37° C. or with an equivalent volume of 50 mM Tris Cl.

Heparitinase-treated and untreated cells were then washed in DMEM without serum and finally resuspended in 0.5 ml DMEM without serum.

Cells were then incubated with a monoclonal antibody to Heparan Sulfate, which was FITC-conjugated (Mab 10E5-FITC, Seikagaku America) (8 μg/ml) for 2 h at 4° C. after that cells were washed and fixed in 0.5% formaldehyde-PBS and then examined by flow cytometry using a Beckman Coulter EPICS XL, following the manufacturer instructions. Data were stored using the SYSTEM II software and analysed using the WinMDI2.8 software.

Cell line and culture conditions: Primary human osteoblasts (HOB) were isolated from explants of trabecular bone dissected from femoral heads. Cells were maintained in Dulbecco's modified Eagle's medium (DMEM) supplemented with 10% FCS, 2 mM glutamine, 1% (v/v) nonessential amino acids and penicillin/streptomycin (100 U/ml and 100 μg/ml, respectively) as described (Verderio et al. 2000 J. Biomed. Mat. Res. 54: 294-304).

Results & Conclusions

Results are shown in FIG. 10.

These data confirm the involvement of heparan sulfate proteoglycans as the receptor to which the tTG/FN complex binds.

### Example C

Changes in tTG-FN-Mediated Actin Assembly in Heparitinase and Chondroitinase-Treated tTG^{-/-} Mefs

Methods

Formation of Stress Fibers in tTG-Deficient Mouse Embryonic Fibroblasts Plated on tTG-FN and Pre-Treated with Heparitinase

A cell suspension of wild type and tTG-deficient mouse embryonic fibroblasts (MEF-TG +/+and MEF-TG−/−, respectively) was prepared from cultures exponentially growing in complete medium with foetal calf serum. Cells were detached from substratum by using 0.25% (v/v) trypsin in 5 mM EDTA, collected into medium containing a ˜7% (v/v) final concentration of FCS, washed with medium without serum and re-suspended at the concentration of 8×10⁴ cells/ml in medium without serum.

The suspension of MEF-TG−/− was pre-incubated with either 30 mU/ml heparitinase I (1000 mU/ml Segakaku America) or 30 mU/ml chondroitinase ABC protease free (1000 mU/ml), for 1 at 37° C. in medium without serum. As a control, both MEF-TG+/+ and MEF-TG−/− were also incubated with an equal volume of 50 mM TrisCl pH 7.0. The treated cells and the control cells were then seeded in 0.79 cm2-wells of chamber slides (2×10⁴/well) previously coated with FN and FN with immobilised tTG (tTG-FN) and allowed to adhere for ˜30 minutes. Cells were rinsed in PBS and fixed using 3.7% paraformaldehyde in PBS pH 7.4. After rinsing 3 times in PBS pH 7.4, cells were permeabilised in 0.1% TritonX-100 in PBS pH 7.4 and then washed in PBS pH 7.4. For detection of stress fibres, fixed and permeabilised cells were blocked in PBS buffer pH 7.4 supplemented with 5% (w/v) dry milk for 30 minutes at ambient temperature and then incubated with FITC-labelled phalloidin at the concentration of 15 μg/ml in blocking buffer for two hours at 37° C. in a humidified chamber. After further washes, coverslips were mounted with Vectashield containing propidium iodide (Vector Laboratories, Peterborough UK) and observed by confocal fluorescent microscopy using a Leica TCSNT confocal laser microscope system. (Leica Lasertechnik, Heidelberg, Germany), equipped with an argon/krypton laser adjusted at 488 nm for fluorescein excitation. For quantification of the formed actin stress fibers consecutive scanning sections (˜2 μm) from the upper to the bottom attachment site of cells were overlaid as an extended focus image and imaged cells (from at least 5 random fields to consider at least 50 cells in control) were scored for stress fiber formation with the aid of the Leica TCSNT (version 1.5-451) image processing menu (Verderio et al. J Biol Chem 2003 278: 42604-42614).

Cell culture conditions: MEFs were maintained in DMEM and Ham's F12 medium supplemented with 10% FCS, 2 mM glutamine and 1% (v/v) nonessential amino acids.

Results & Conclusions

Results show that tTG-FN complexes restores the ability of tTG-deficient mouse embryonic fibroblasts (MEFs) to form actin stress fibers (see FIG. 11). This restorative effect is lost by pre-incubation of MEFs with heparatinase, but not by pre-incubation with chondroitinase.

These data further confirm the involvement of tTG-FN complexes, and heparan sulfate proteoglycans, in actin cytoskeleton formation.

### Example D

Tissue Transglutaminase Bound to Fibronectin Supports RGD Independent-Cell Adhesion of BRIN-BD11 Cells

Methods

A novel insulin-secreting cell line (BRIN-BD11), which was established after electrofusion of RINm5F cells with New England Deaconess Hospital rat pancreatic islet cells, was grown confluent in RPMI 1640 media, supplemented with 10% FCS, 200 u/ml Penicillin, 200 ug/ml Streptomycin and L-Glutamine at 2 mM. The wells of microtitre plates were coated with FN (5 μg/ml) overnight at 4° C. The assigned wells were washed once with 50 mM Tris-HCl, pH 7.4 and then incubated with 20 μg/ml tTG solution (dissolved in PBS containing 2 mM EDTA pH 7.4) for 1 hour at 37° C. Meanwhile, exponentially growing cells were trypsinised and collected into medium containing approximately 7% (v/v) foetal calf serum. After a five-minute centrifugation at 300×g the cell pellet was washed twice in serum free medium to remove the traces of serum proteins. A cell suspension of 2×10⁵ cell/ml was prepared, and incubated with GRGDTP synthetic peptide at concentrations of 5 μg/ml and 10 μg/ml for 20 minutes at 37° C., in a 5% (v/v) CO2, 95% (v/v) air atmosphere. The cells (2×10⁴ cells/well) were seeded on FN or FN/tTG matrices in the presence of GRGDTP synthetic peptide and allowed to attached overnight at 37° C., in a 5% (v/v) CO2, 95% (v/v) air atmosphere. Upon cell adhesion and spreading, the medium was carefully removed and wells were gently washed once with PBS, pH 7.4. Cells were then fixed with 100 μl of 3.7% (w/v) paraformaldehyde dissolved in PBS PH 7.4 for 15 minutes at room temperature and washed twice with PBS, pH 7.4. Following fixation, cells were permeabilised with 100 μl of 0.1% (v/v) Triton-X in PBS, pH 7.4 for 15 minutes at room temperature. The wells were then washed twice with PBS, pH 7.4. To visualise the attached cells, a two-step staining process was employed to stain both cytoplasm and nucleus. Following the cell permeablisation, 100 μl of May-Grunwald stain (Sigma) was added to the wells to stain the cell cytoplasm for 15 minutes at room temperature. The stain was then removed and wells were washed once with PBS, pH 7.4. The wells were then incubated with 100 μl of 5% (v/v) Giemsa stain (Sigma) in dH₂O to co-stain the nucleus for 20 minutes at room temperature. The plate finally washed twice with dH₂O and left to dry. Digital images of six non-overlapping fields covering the centre of the well were acquired using a video digital camera Olympus DP10. The cell attachment and spreading were quantified using the Scion image analysis programme, which is developed at the National Institute of Health (Washington DC, USA). The number of cells per image was assessed through threshold and particle analysis settings with a minimum particle size of 50 pixels. Spread cells were discriminated from non-spread by their two-colour appearance (dark purple nucleus, pink haled cytoplasm). These were quantified by density slicing and particle analysis settings. The spread cell particles were selectively highlighted through the adjustment of the Lut intensity.

Results & Discussion

Cell attachment and cell spreading was measured after seeding of BRIN-BD11 (clonal b-cells) onto fibronectin (FN) in the absence or presence of tissue transglutaminase (tTG-FN) (see FIG. 12). Cells were pre-incubated with excess RGD peptide (+RGD) to inhibit the RGD-dependent integrin receptors (RAD peptide was used as control peptide).

Cell attachment and spreading on FN was decreased by incubation with excess RGD peptide but was restored by the association of tTG to FN. Similarly, the inhibition of actin stress fibre assembly by function-blocking anti-integrin a5 and b1 antibodies was partly overcome by binding of tTG to FN, which led to the formation of unique focal adhesions (not shown). These data indicate that tTG in complex with FN supports cell adhesion via an RGD-independent mechanism in β-cells as well as in fibroblasts and HOB cells (data not shown).

### Example E

Pharmaceutical Formulations

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen-free.

The following examples illustrate pharmaceutical formulations according to the invention in which the active ingredient is a compound of the invention (e.g. Compound 2).

### Example A

Tablet

| | | |
|---|---|---|
| Active ingredient | 100 | mg |
| Lactose | 200 | mg |
| Starch | 50 | mg |
| Polyvinylpyrrolidone | 5 | mg |
| Magnesium stearate | 4 | mg |
| | 359 | mg |

Tablets are prepared from the foregoing ingredients by wet granulation followed by compression.

### Example B

Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |

| | |
|---|---|
| pH adjusted to | 7.5 |

### Example C

Tablet Formulations

The following formulations A and B are prepared by wet granulation of the ingredients with a solution of povidone, followed by addition of magnesium stearate and compression.

Formulation A

| | mg/tablet | mg/tablet |
|---|---|---|
| (a) Active ingredient | 250 | 250 |
| (b) Lactose B.P. | 210 | 26 |
| (c) Povidone B.P. | 15 | 9 |
| (d) Sodium Starch Glycolate | 20 | 12 |
| (e) Magnesium Stearate | 5 | 3 |
| | — | |
| | 500 | 300 |

Formulation B

| | mg/tablet | mg/tablet |
|---|---|---|
| (a) Active ingredient | 250 | 250 |
| (b) Lactose | 150 | — |
| (c) Avicel PH 101 ® | 60 | 26 |
| (d) Povidone B.P. | 15 | 9 |
| (e) Sodium Starch Glycolate | 20 | 12 |
| (f) Magnesium Stearate | 5 | 3 |
| | — | |
| | 500 | 300 |

Formulation C

| | mg/tablet |
|---|---|
| Active ingredient | 100 |
| Lactose | 200 |
| Starch | 50 |
| Povidone | 5 |
| Magnesium stearate | 4 |

The following formulations, D and E, are prepared by direct compression of the admixed ingredients. The lactose used in formulation E is of the direction compression type.

Formulation D

| | mg/capsule |
|---|---|
| Active Ingredient | 250 |
| Pregelatinised Starch NF15 | 150 |
| | 400 |

Formulation E

| | mg/capsule |
|---|---|
| Active Ingredient | 250 |
| Lactose | 150 |
| Avicel ® | 100 |
| | 500 |

Formulation F (Controlled Release Formulation)

The formulation is prepared by wet granulation of the ingredients (below) with a solution of povidone followed by the addition of magnesium stearate and compression.

| | mg/tablet |
|---|---|
| Active Ingredient | 500 |
| Hydroxypropylmethylcellulose | 112 |
| (Methocel K4M Premium) ® | |
| Lactose B.P. | 53 |
| Povidone B.P.C. | 28 |
| Magnesium Stearate | 7 |
| | 700 |

Drug release takes place over a period of about 6-8 hours and was complete after 12 hours.

### Example D

Capsule Formulations

Formulation A

A capsule formulation is prepared by admixing the ingredients of Formulation D in Example C above and filling into a two-part hard gelatin capsule. Formulation B (infra) is prepared in a similar manner.

Formulation B

| | mg/capsule |
|---|---|
| Active ingredient | 250 |
| Lactose B.P. | 143 |
| Sodium Starch Glycolate | 25 |
| Magnesium Stearate | 2 |
| | 420 |

Formulation C

| | mg/capsule |
|---|---|
| Active ingredient | 250 |
| Macrogol 4000 BP | 350 |
| | 600 |

Capsules are prepared by melting the Macrogol 4000 BP, dispersing the active ingredient in the melt and filling the melt into a two-part hard gelatin capsule.

Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| Active ingredient | 250 |
| Microcrystalline Cellulose | 125 |
| Lactose BP | 125 |
| Ethyl Cellulose | 13 |
| | 513 |

### Example E

Injectable Formulation

| | |
|---|---|
| Active ingredient | 0.200 g |

Sterile, pyrogen free phosphate buffer (pH 7.0) to 10 ml

The active ingredient is dissolved in most of the phosphate buffer (35-40° C.), then made up to volume and filtered through a sterile micropore filter into a sterile 10 ml amber glass vial (type 1) and sealed with sterile closures and overseals.

### Example F

Intramuscular Injection

| | | |
|---|---|---|
| Active ingredient | 0.20 | g |
| Benzyl Alcohol | 0.10 | g |
| Glucofurol 75 ® | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### Example G

Syrup Suspension

| | | |
|---|---|---|
| Active ingredient | 0.2500 | g |
| Sorbitol Solution | 1.5000 | g |
| Glycerol | 2.0000 | g |
| Dispersible Cellulose | 0.0750 | g |
| Sodium Benzoate | 0.0050 | g |
| Flavour, Peach 17.42.3169 | 0.0125 | ml |
| Purified Water q.s. to | 5.0000 | ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### Example H

Suppository

| | mg/suppository |
|---|---|
| Active ingredient (63 μm)* | 250 |
| Hard Fat, BP (Witepsol H15 - Dynamit Nobel) | 1770 |
| | 2020 |

*The active ingredient is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### Example I

Pessaries

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### Example J

Creams and Ointments

(see Remington: The Science and Practise of Pharmacy, 19^{th} ed., The Philadelphia College of Pharmacy and Science, ISBN 0-912734-04-3)

Therapeutic use of a Compound of the Invention in the Treatment of a Proliferative Disorder

An amount of a compound of the invention is dissolved in sterile, non-pyrogenic water or isotonic saline. The solution is administered to a patient suffering from a proliferative disorder, either directly to the tumour site or systemically.

Preferably, the solution is administered at regular intervals (e.g. daily, twice weekly, weekly or monthly) for a prolonged period, such that the symptoms associated with the proliferative disorder (e.g. tumour size) are eased or are prevented from worsening.

### REFERENCES

Achyuthan, K. E., R. J. Goodell, J. R. Kennedye, K. N. Lee, A Henley, J. R. Stiefer and P. J. Birkbickler. 1995. Immunochemical analyses of human plasma fibronectin-cytosolic transglutaminase interactions. J. Immunol Methods. 180:69-79.
Aeschlimann, D. and V. Thommazy. 2000. Protein crosslinking in assembly and remodelling of extracellular matrices: the role of transglutaminases. Connect. Tissue Res. 41:1-27.
Akimov, S. S., D. Krylov, L. F. Fleischman, and A. M. Belkin. 2000. Tissue transglutaminase is an integrin-binding adhesion coreceptor for fibronectin. J. Cell Biol., 148:825-838.
Antonyak, M. A., U. S. Singh, D. A. Lee, J. E. Boehm, C. Combs, M. M. Zgola, R. L. Page, and R. A. Cerione. 2001. Effects of tissue transglutaminase on retinoic acid-induced cellular differentiation and protection against apoptosis. J. Biol. Chem. 276:36 33582-7.
Aplin, A. E., A. K. Howe, and R. L. Juliano. 1999. Cell adhesion molecules, signal transduction and cell growth. Curr. Opin. Cell. Biol. 11:6 737-44.
Attwell, S., C. Roskelley, and S. Dedhar. 2000. The integrin-linked kinase (ILK) suppresses anoikis. Oncogene 19:33 3811-5.
Balklava, Z., E. Verderio, R. Collighan, S. Gross, J. Adams, and M. Griffin. 2002. Analysis of tissue transglutaminase function in the migration of Swiss 3T3 fibroblasts: the active-state conformation of the enzyme does not affect cell motility but it is important for its secretion. J. Biol. Chem. 277:16567-16575.
Bass & Humphries, 2002. Cytoplasmic interactions of syndecan-4 orchestrate adhesion receptor and growth factor receptor signalling. Biochem. J. 368: 1-15.
Belkin, A. M., S. S. Akimov, L. S. Zaritskaya, B. I. Ratnikov, E. I. Deryugina, and A. Y. Strongin. 2001. Matrix-dependent proteolysis of surface transglutaminase by membrane-type metalloproteinase regulates cancer cell adhesion and locomotion. J. Biol. Chem. 276:18415-18422.
Bergamini, C. M., and M. Signorini. 1993. Studies on tissue transglutaminases: interaction of erythrocyte type-2 transglutaminase with GTP. Biochem. J. 291 (Pt 1): 37-9.
Boudreau, N., C. J. Sympson, Z. Werb, and M. J. Bissell. 1995. Suppression of ICE and apoptosis in mammary epithelial cells by extracellular matrix. Science. 267:891-3.
Brooks, P. C. A. M. Montgomery, M. Rosenfeld, R. A. Reisfeld, T. Hu, G. Klier, and D. A. Cheresh. 1994. Integrin Antagonists Promote Tumor Regression by Inducing Apoptosis of Angiogenic Blood Vessels.Cell 79:1157.
Dalton, B. A., C. D. McFarland, P. A. Underwood, and J. G. Steele. 1995. Role of the heparin binding domain of fibronectin in attachment and spreading of human bone-derived cells. J. Cell Sci. 108 ( Pt 5): 2083-92.
Davis, G. E., K. J. Bayless, M. J. Davis, and G. A. Meininger. 2000. Regulation of tissue injury responses by the exposure of matricryptic sites within extracellular matrix molecules. Am. J. Pathol. 156:5,1489-98.
Dedhar, S. 2000. Cell-substrate interactions and signalling through ILK. Curr. Opin. Cell Biol. 12:2 250-6.
De Laurenzi, V. and G. Melino. 2001. Gene disruption of tissue transglutaminase. Mol. Cell Biol. 21:148-155.
Freund, K. F., K. P. Doshi, S. L. Gaul, D. A. Claremon, D. C. Remy, J. J. Baldwin, S. M. Pitzenberger, and A. M. Stem. 1994. Transglutaminase inhibition by 2-[(2-oxopropyl)thio]imidazolium derivatives: mechanism of Factor XIIIa inactivation. Biochemistry, 33:10109-10119.
Frisch, S. M. and R. A. Screaton. 2001. Anoikis mechanisms. Curr. Opin. Cell Biol. 13:5 555-62.
Gallo, R., C. Kim, R. Kokenyesi, N. S. Adzik and M. Bernfield. 1996. Syndecans-1 and 4 induced during wound repair of neonatal but not fetal skin. J. Invest. Dermat. 107:676-683.
Gaudry, C. A., E. Verderio, D. Aeschlimann, A. Cox, C. Smith, and M. Griffin. 1999. Cell surface localisation of tissue transglutaminase is dependent on a fibronectin-binding site in its N-terminal beta-sandwich domain. J. Biol. Chem. 274:30707-30714.
Gentile, V., V. Thomazy, M. Piacentini, L. Fesus, and P. J. Davies. 1992. Expression of tissue transglutaminase in Balb-C 3T3 fibroblasts: effects on cellular morphology and adhesion. J. Cell Biol. 119:463-474.
Gronthos, S., K. Stewart, S. E. Graves, S. Hay, and P. J. Simmons. 1997. Integrin expression and function on human osteoblast-like cells. J. Bone Miner. Res. 12:8 1189-97.
Griffin, M., R. Casadio, and C. Bergamini. 2002. Transglutamninases, Nature's Biological Glues. J. Biochem. In press.
Griffin, M. and E. Verderio. 2000. Tissue transglutaminase in cell death. In Programmed Cell Death in Animals and Plants. J. A. Bryant, S. G. Hughes, and J. M. Garland, editors. BIOS Sci. Publ. Oxford. 223-240pp.
Gschwendt, M., S. Dieterich, J. Rennecke, W. Kittstein, H. J. Muller and F. J. Johannes. 1996. Inhibition of protein kinase C mu by various inhibitors. Differentiation from protein kinase c isoenzymes. FEBS Lett. 392(2):77-80.
Hadden, H. L. and C. A. Henke. 2000. Induction of lung fibrosis apoptosis by soluble fibronectin peptides. Am. J. Respir. Crit. Care Med. 162:1553-1560.
Haroon, Z. A., J. M. Hettasch, T. S. Lai, M. W. Dewhirst, and C. S. Greenberg. 1999. Tissue transglutaminase is expressed, active, and directly involved in rat dermal wound healing and angiogenesis. FASEB J. 13:1787-1795.
Hasegawa, G. and Y. Saito. 2002. Tissue-type transglutaminase has protein disulfide isomerase activity. Minerva Biotecnologica. 192pp.
Heath, D. J., S. Downes, E. Verderio, and M. Griffin. 2001. Characterisation of tissue transglutaminase in human osteoblast-like cells. J. Bone Miner. Res. 8:1477-85.
Heath, D. J., P. Christian, and M. Griffin. 2002. Involvement of tissue transglutaminase in the stabilisation of biomaterial/tissue interfaces important in medical devices. Biomaterials. 23:6 1519-26.
Hocking, D. C., J. Sottile, and K. J. Langenbach. 2000. Stimulation of integrin-mediated cell contractility by fibronectin polymerization. J. Biol. Chem. 275:14 10673-82.
Humphries, M. J. 2000. Integrin cell adhesion receptors and the concept of agonism. Trends Pharmacol. Sci. 21:1 29-32.
Huser, M., J. Luckett, A. Chiloeches, K. Mercer, M. Iwobi, S. Giblett, X. M. Sun, J. Brown, R. Marais, and C. M. E. K. Pritchard. 2001. Kinase activity is not necessary for Raf-l function. EMBO J. 20:8 1940-51.
Jeong, J. M., S. N. Murthy, J. T. Radek, and L. Lorand. 1995. The fibronectin-binding domain of transglutaminase. J. Biol. Chem. 10:5654-8.
Jeong, J., I. Han, Y. Lim, J. Kim, I. Parks, A. Wood, J. R. Couchman and E.S. Oh. 2001. Rat embryo fibroblasts require both the cell-binding and heparin binding domains of fibronectin for survival. Biochem. J. 356:231-537.
Johnson, T. S., N. J. Skill, A. M. El Nahas, S. D. Oldroyd, G. L. Thomas, J. A. Douthwaite, J. L. Haylor, and M. Griffin. 1999. Transglutaminase transcription and antigen translocation in experimental renal scarring. J. Am. Soc. Nephrol. 10:2146-2157.
Johnson, T. S., C. I. Schofield, J. Parry and M. Griffin. 1998. Induction of tissue transglutaminase by dexamethasone: its correlation to receptor number and transglutaminase-mediated cell death in a series of malignant hamster fibrosarcomas. Biochem. J. 331:105-112.
Jones, R. A., B. Nicholas, S. Mian, P. J. Davies, and M. Griffin. 1997. Reduced expression of tissue transglutaminase in a human endothelial cell line leads to changes in cell spreading, cell adhesion and reduced polymerisation of fibronectin. J. Cell Sci. 110: 2461-2472.
Kapila, Y. L., S. Wang, P. Dazin, E. Tafolla, and M. J. Mass. 2002. The Heparin-binding domain and V region of fibronectin regulate apoptosis by suppression of p53 and c-myc in human primary cells. J. Biol. Chem. 277:10 8482-91.
Layemi, M., S. Demignot, L. Borge, S. Thenet-Gauci, and M. Adolphe. 1997. The use of fluorescein cadaverine for detecting amine acceptor protein substrates accessible to active transglutaminase in living cells. Histochem. J. 29:593-606.
Leblanc, A., N. Day, A. Menard, and J. W. Keillor. Guinea pig liver transglutaminase: A modified purification procedure affording enzyme with superior activity in greater yield. Protein Expr. Purif. 17:1 89-95.
LeMosy, E. K., H. P. Erickson, W. F. Beyer, J. T. Radek, J. M. Jeong, S. N. Murthy, and L. Lorand. 1992. Visualization of purified fibronectin-transglutaminase complexes. J. Biol. Chem. 267:11 7880-5.
Lorand, L., J. E. Dailey, and P. M. Turner. 1988. Fibronectin as a carrier for the transglutaminase from human erythrocytes. Proc. Natl. Acad. Sci. USA. 85:1057-1059.
Martinez, J., D. G. Chalupowiucz, R. K. Roush, A. Sheth and C. Barsigian. 1994. Transglutaminase-mediated processing of fibronectin by endothelial cell monolayers. Biochemistry. 33:2538-2545.
McCawley, L. J., and L. M. Matrisian. 2001. Matrix metalloproteinases: they're not just for matrix anymore! Curr. Opin. Cell Biol. 13:5 53440.
McGill, G., A. Shimamura, R. C. Bates, R. E. Savage, and D. E. Fisher. 1997. Loss of matrix adhesion triggers rapid transformation-selective apoptosis in fibroblasts. J. Cell. Biol. 138:4 901-11.
Melino, G., C. J. Thiele, R. A. Knight, and M. Piacentini. 1997. Retinoids and the control of growth/death decisions in human neuroblastoma cell lines. J. Neurooncol. 31:65-83.
Mikula, M., M. Schreiber, Z. Husak, L. Kucerova, J. Ruth, R. Wieser, K. Zatloukal, H. Beug, E. F. Wagner, and M. Baccarini. 2001. Embryonic lethality and fetal liver apoptosis in mice lacking the c-raf-1 gene. EMBO J. 20:8 1952-62.
Nanda, N., S. E. Iismaa, W. A. Owens, A. Husain, F. Mackay, and R. M. Graham. 2001. Targeted inactivation of Gh/tissue transglutaminase II. J. Biol. Chem. 276:23 20673-8.
Nagy, L., M. Saydak, N. Shipley, S. Lu, J. P. Basilion, Z. H. Yan, P. Syka, R.A.S. Chandraratna, J.P. Stein, R.A. Heyman, and P. J. A. Davies. 1996.
Identification and characterisation of a versatile retinoid response element (retinoic acid receptor response element-retinoid X receptor response element) in the mouse tissue transglutaminase gene promoter. J. Biol. Chem. 271:4355-4365.
Oh, E. S., A. Woods, and J. R. Couchman. 1997. Syndecan-4 proteoglycan regulates the distribution and activity of protein kinase C. J. Biol. Chem. 272:13 8133-6.
Pereira, M, B. J. Rybarczyk, T. M. Odrljin, D. C. Hocking, J. Sottile, and P. J. Simpson-Haidaris. 2002. The incorporation of fibrinogen into extracellular matrix is dependent on active assembly of a fibronectin matrix. J. Cell Sci. 115:Pt 3 609-17.
Piacentini, M., M. G. Farrace, P. Matarrese, F. Ciccosanti, L. Falasca, C. Rodolfo, A. M. Giammarioli, E. Verderio, M. Griffin, and W. Malorni. 2002. Transglutaminase overexpression sensitises neuronal cell lines to apoptosis by increasing mitochondrial membrane potential and cellular oxidative stress. J. Neurochem. 81:1061-1072.
Radek, J. T., J. M. Jeong, S. N. Murthy, K. C. Ingham, and L. Lorand. 1993. Affinity of human erythrocyte transglutaminase for a 42-kDa gelatin-binding fragment of human plasma fibronectin. Proc. Natl. Acad. Sci. USA. 90:8 3152-6.
Ritter, S. J. and P. J. Davies. 1998. Identification of a transforming growth factor-beta1/bone morphogenetic protein 4 (TGF-beta1/BMP4) response element within the mouse tissue transglutaminase gene promoter. J. Biol. Chem. 21:12798-806.
Ishiguro, K., K. Kadomatsu, T. Kojina, H. Muramatsu, S. Tsuzuki, E. Nakamura, K. Kusugami, H. Saito, and T. Muramatsu. 2000. Syndecan-4 deficiency impairs focal adhesion formation only under restricted conditions. J. Biol. Chem. 275:8 5249-52
Schlaepfer, D. D., C. R. Hauck, and D. J. Sieg. 1999. Signalling through focal adhesion kinase. Prog. Biophys. Mol. Biol. 71:3-4 435-78.
Sechler, J. L., and J. E. Schwarzbauer. 1998. Control of cell cycle progression by fibronectin matrix architecture. J. Biol. Chem. 273:40 25533-6.
Sechler, J. L., A. M. Cumiskey, D. M. Gazzola, and J. E. Schwarzbauer. 2000. A novel RGD-independent fibronectin assembly pathway initiated by alpha4beta1 integrin binding to the alternatively spliced V region. J. Cell Sci. 113 (Pt 8): 1491-8.
Sharma, A., J. A. Askari, M. J. Humphries, E. Y. Jones, and D. I. Stuart. 1999. Crystal structure of a heparin- and integrin-binding segment of human fibronectin. EMBO J. 18:6 1468-79.
Sonoda, Y., Y. Matsumoto, M. Funakoshi, D. Yamamoto, S. K. Hanks, and T. Kasahara. 2000. Anti-apoptotic role of focal adhesion kinase (FAK). Induction of inhibitor-of-apoptosis proteins and apoptosis suppression by the overexpression of FAK in a human leukemic cell line, HL-60. J. Biol. Chem. 275:21 16309-15.
Sottile, J., D. C. Hocking, and K. J. Langenbach. 2000. Fibronectin polymerization stimulates cell growth by RGD-dependent and -independent mechanisms. J. Cell Sci. 113 Pt 23:4287-99.
Takahashi, H., T. Isobe, S. Horibe, J. Takagi, Y. Yokosaki, D. Sheppard, and Y. Saito. 2000. Tissue transglutaminase, coagulation factor XIII, and the pro-polypeptide of von Willebrand factor are all ligands for the integrins alpha 9beta 1 and alpha 4beta 1. J. Biol. Chem. 275:31 23589-95.
Tumova, S., A. Woods, and J. R. Couchman. 2000. Heparan sulfate chains from glypican and syndecans bind the Hep II domain of fibronectin similarly despite minor structural differences. J. Biol. Chem. 275:9410-7.
Upchurch, H. F., E. Conway, M. K. Patterson Jnr, P. I. Birkbichler, and M. D. Maxwell. 1987. Cellular transglutaminase has affinity for extra-cellular matrix. In vitro Cellular Developmental Biology, 23:795-800.
Verderio, E., B. Nicholas, S. Gross, and M. Griffin. 1998. Regulated expression of tissue transglutaminase in Swiss 3T3 fibroblasts: effects on the processing of fibronectin, cell attachment and cell death. Exp. Cell Res. 239:119-138.
Verderio, E., C. A. Gaudry, S. Gross, C. Smith, S. Downes, and M. Griffin. 1999. Regulation of cell surface tissue transglutaminase: effects on matrix storage of latent transforming growth factor ®-binding protein-1. J. Histochem Cytochem., 47:1417-1432.
Verderio, E., A. Coombes, R. A. Jones, X. Li, D. Heath, S. Downes, and M. Griffin. 2000. Role of the crosslinking enzyme tissue transglutaminase in the biological recognition of synthetic biodegradable polymers. J. Biomedical Mat. Res. 54:294-304.
Wilcox-Adelman, S. A., F. Denhez, and P. F. Goetinck. 2002. Syndecan-4 modulates focal adhesion kinase phosphorylation. J. Biol. Chem. 277:36 32970-7.
Woods, A. and J. R. Couchman. 2001. Syndecan-4 and focal adhesion function. Curr. Opin. Cell Biol. 13:5 578-83.
Woods, A., J. R. Couchman, S. Johansson, and M. Hook. 1986. Adhesion and cytoskeletal organisation of fibroblasts in response to fibronectin fragments. EMBO J 5:4 665-70.
Woods, A. 2001. Syndecans: transmembrane modulators of adhesion and matrix assembly. J. Clin. Invest. 107:8 935-41.

1. A method for identifying a drug-like compound, or lead compound for the development of a drug-like compound, which compound is capable of modulating cellular adhesion and/or cell survival comprising the step of testing said compound for an ability to modulate the binding of a complex of tissue transglutaminase and fibronectin to a heparan sulfate containing receptor.

2. A method according to claim 1 wherein the method is for identifying a drug-like compound or lead compound for the development of a drug-like compound for use in the treatment of proliferative disorders.

3. A method according to claim 1 wherein the method is for identifying a drug-like compound or lead compound for the development of a drug-like compound for use modulating wound healing.

4. A method according to claim 1 comprising testing said compound for an ability to increase the binding of a complex of tissue transglutaminase and fibronectin to a heparan sulfate containing receptor.

5. A method according to claim 1 comprising testing said compound for an ability to decrease the binding of a complex of tissue transglutaminase and fibronectin to a heparan sulfate containing receptor.

6. A method according to claim 1 wherein testing said compound for an ability to modulate the binding of a complex of tissue transglutaminase and fibronectin to a heparan sulfate containing receptor comprises testing said compound for an ability to modulate the functional consequences of binding of a complex of tissue transglutaminase and fibronectin to a heparan sulfate containing receptor.

7. A method according to claim 1 comprising the following steps:
(a) contacting a tissue transglutaminase (tTG) with fibronectin (FN) to form a tTG/FN complex;
(b) exposing the tTG/FN complex of step (a) to a compound to be tested;
(c) exposing the treated complex of step (b) to a heparan sulfate containing receptor; and
(d) measuring the binding of the treated complex to the heparan sulfate containing receptor and/or a functional marker of such binding.

8. A method according to claim 1 comprising the following steps:
(a) contacting tTG with FN to form a tTG/FN complex;
(b) exposing a heparan sulfate containing receptor to a compound to be tested;
(c) exposing the tTG/FN complex of step (a) to the treated heparan sulfate containing receptor of step (b); and
(d) measuring the binding of the treated complex to the heparan sulfate containing receptor and/or a functional marker of such binding.

9. A method according to claim 1 comprising the following steps:
(a) contacting tTG with FN to form a tTG/FN complex;
(b) exposing the tTG/FN complex of step (a) to a heparan sulfate containing receptor;
(c) exposing the tTG/FN complex and heparan sulfate containing receptor of step (b) to a compound to be tested; and
(d) measuring the binding of the treated complex to the heparan sulfate containing receptor and/or a functional marker of such binding.

10. A method according to claim 1 wherein the tissue transglutaminase is a mammalian tissue transglutaminase.

11. A method according to claim 10 wherein the tissue transglutaminase is a human tissue transglutaminase.

12. A method according to claim 1 wherein the tissue transglutaminase and/or fibronectin is a recombinant protein.

13. A method according to claim 1 wherein the tissue transglutaminase and/or fibronectin are derived from cells capable of expressing and secreting tTG and FN.

14. A method according to claim 1 wherein the tissue transglutaminase and/or fibronectin is a naturally occurring protein.

15. A method according to claim 1 wherein the tissue transglutaminase and/or fibronectin is a variant of a wildtype tissue transglutaminase or fibronectin, respectively, which variant has an ability to form tTG/FN complexes capable of binding to a heparan sulfate containing receptor.

16. A method according to claim 1 wherein the compound to be tested is used at a range of doses and/or for various incubation times.

17. A method according to claim 1 wherein heparan sulfate containing receptor is syndecan-4, or a fragment thereof which has an ability to bind to tTG/FN complexes.

18. A method according to claim 1 wherein the heparan sulfate containing receptor is expressed on the surface of a cell.

19. A method according to claim 18 wherein the cell is selected from the group consisting of a primary human osteoblasts, a Swiss 3T3 fibroblasts and human bladder carcinoma ECV304 cells.

20. A method according to claim 18 wherein the cell is transfected such that a heparan sulfate containing receptor is expressed on the surface of a cell.

21. A method according claim 7 wherein, in step (c), RGD-dependent binding is inhibited by incubating the tTG/FN complex and heparan sulfate containing receptor in the presence of an RGD-containing peptide.

22. A method according to claim 7 wherein the method comprises comparing the binding measured in step (d) to that measured in the absence of treatment with a compound to be tested, a difference in binding being indicative of modulation of cellular adhesion and/or cell survival.

23. A method according to claim 7 wherein step (d) comprises measuring RGD-independent binding.

24. A method according to claim 7 wherein step (d) is performed using an ELISA.

25. A method according to claim 7 wherein step (d) comprises measuring a functional marker of the binding of the tTG/FN complex to the heparan sulfate containing receptor.

26. A method according to claim 25 wherein the functional marker is selected from cell attachment, cell spreading, formation of focal adhesion structures, PKCα activity and FAK activity.

27. A method according to claim 1 wherein the method is capable of high throughput.

28. A method according to claim 7 wherein the tTG/FN complex is immobilised.

29. A method according to claim 22 wherein the compound is identified as a positive modulator if it increases binding of a complex of tissue transglutaminase and fibronectin to a heparan sulfate containing receptor by at least 10% compared to binding in a control sample which has not been treated with the compound to be tested,

30. A method according to claim 29 wherein the compound is identified as a positive modulator if it increases binding of a complex of tissue transglutaminase and fibronectin to a heparan sulfate containing receptor by at least 20%, 30%, 40%, 50%, 60%, 70%, 80%, 90%, 100%, 150%, 200%, 300%, 400%, 500% or 1000% compared to untreated controls.

31. A method according to claim 22 wherein the compound is identified as a negative modulator if it decreases binding of a complex of tissue transglutaminase and fibronectin to a heparan sulfate containing receptor by at least 10% compared to binding in a control sample which has not been treated with the compound to be tested.

32. A method according to claim 31 wherein the compound is identified as a negative modulator if it decreases binding of a complex of tissue transglutaminase and fibronectin to a heparan sulfate containing receptor by at least 20%, 30%, 40%, 50%, 60%, 70%, 80%, 90%, 95%, 96%, 97%, 98%, 99% or 100% compared to untreated controls.

33. A method according to claim 7 wherein the method further comprises a pre-screening step, prior to step ‘a’), of testing a prospective test compound to determine if it binds to tissue transglutaminase and/or fibronectin, either individually or in a complex (in the absence of a heparan sulfate containing receptor), and/or if it binds to a heparan sulfate containing receptor (in the absence of a complex of tissue transglutaminase and fibronectin) and selecting prospective test compounds which exhibit such binding as compounds for further testing.

34. A method according to claim 1 wherein the results of the methods are furnished in an intelligible format.

35. A method according to claim 34 wherein the results are recorded or stored on an information carrier.

36. A kit of parts for use in the method according to claim 1, the kit comprising:
(a) a substrate upon which tTG/FN complexes are immobilised or means for producing such as surface;
(b) a heparan sulfate containing receptor; and
(c) means for detecting the binding of tTG/FN complexes to the heparan sulfate containing receptor.

37. A kit according to claim 36 wherein the kit comprises a surface upon which tTG/FN complexes are immobilised.

38. A kit according to claim 36 wherein the kit comprises a surface to which tTG/FN complexes may be adhered and cells capable of expressing such complexes.

39. A kit according to claim 36 wherein the surface is a surface of a well in a single- or multi-well plate.

40. A compound identifiable by a method according to claim 1.

41. A pharmaceutical formulation comprising a compound according to claim 40 in admixture with a pharmaceutically or veterinarily acceptable adjuvant, diluent or carrier.

42. (canceled)

43. (canceled)

44. (canceled)

45. (canceled)

46. (canceled)

47. A method for treating a patient, either prophylactically or curatively, having a proliferative disorder, the method comprising administering to the patient a compound according to claim 40.

48. (canceled)

49. (canceled)

50. (canceled)

51. (canceled)

52. (canceled)

53. (canceled)

54. (canceled)

55. (canceled)

56. (canceled)
